(19) United States
(12) Patent Application Publication
(10) Pub. No.: US 2019/0038602 A1
(43) Pub. Date: Feb. 7, 2019

(54) GRANZYME B INHIBITOR FORMULATIONS AND METHODS FOR THE TREATMENT OF BURNS

(71) Applicants: viDA Therapeutics, Inc., North Vancouver, BC (CA); The University of British Columbia, Vancouver, BC (CA)

(72) Inventors: David Granville, Port Coquitlam (CA); Yue Shen, Burnaby (CA); Maria Fernanda Zuluaga Estrada, West Vancouver (CA); Dale R. Cameron, Richmond (CA); Anthony S.K. Boey, Coqutilam (CA)

(73) Assignees: viDA Therapeutics, Inc., North Vancouver, BC (CA); The University of British Columbia, Vancouver, BC (CA)

(21) Appl. No.: 16/075,039

(22) PCT Filed: Feb. 3, 2017
(86) PCT No.: PCT/CA2017/050126

§ 371 Date: Aug. 2, 2018

Related U.S. Application Data
(60) Provisional application No. 62/290,862, filed on Feb. 3, 2016.

(51) Int. Cl. A61K 31/4178; A61K 47/10; A61K 47/32; A61K 9/00; A61P 17/02

(52) U.S. Cl. CPC A61K 31/4178; A61K 47/10; A61P 17/02; A61K 9/0019; A61K 9/0014; A61K 47/32

(57) ABSTRACT

Formulations for treating burns and burn wound healing comprising a Granzyme B inhibitor and a pharmaceutically acceptable carrier, and methods for treating burns and for burn wound healing using the formulations.

### CROSS-REFERENCE TO RELATED APPLICATION

This application claims the benefit of U.S. Application No. 62/290,862, filed Feb. 3, 2016, expressly incorporated herein by reference in its entirety.

### BACKGROUND OF THE INVENTION

Burn trauma is a type of injury that can be caused by heat, freezing, electricity, chemicals, radiation, or friction. Burn trauma is highly variable based on the tissue affected, the severity of the burn, and resultant complications. Beyond physical complications, burns can also result in severe physiological and emotional distress due to long-term hospitalization, scarring, and deformity.

The extent of burn injury is related to wound depths and internationally classified as first (superficial), second (superficial, partial thickness or superficial, deep thickness) or third degree (full thickness). The depth of burn wound evolves with time, especially with partial thickness wounds. Wounds that start as mild/moderate second-degree burns may progress to deep partial or third-degree burns over 2-4 days post-burn injury. Burn wounds can be classified into 3 distinct areas: (1) zone of necrosis—this area is the dead tissue that is unsalvageable, (2) zone of stasis—cell death in the zone of stasis has been thought to be responsible for the progression of wounds, and (3) zone of hyperemia—viable tissue that usually recovers. Because of this unique pathophysiology, burn patients with partial thickness burn wounds must be evaluated for depth of the wound periodically. As a rule, partial thickness burns that are predicted not to heal by 3 weeks should be excised and grafted. As burn progression leading to the requirement for more advanced therapy and/or engraftment is thought to occur in the zone of stasis, agents that can prevent the expansion of the zone of stasis and wound severity would revolutionize the treatment of burns.

Despite the advances in development of burn treatments, a need for exists for new treatments including and improved formulations for use in treating burns and to improve burn wound healing. The present invention seeks to fulfill this need and provides further related advantages.

### SUMMARY OF THE INVENTION

The present invention provides formulations and methods for the treatment of burns and burn wound healing. The formulations include a Granzyme B inhibitor.

In one aspect, the invention provides formulations that include a Granzyme B inhibitor compound effective for treating burns and for burn wound healing. In one embodiment, the invention provides a formulation for burn wound healing, comprising 4-(((2S,3S)-1-((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)amino)-4-oxobutanoic acid or a pharmaceutically acceptable salt thereof in combination with a pharmaceutically acceptable carrier.

In another aspect of the invention, methods for treating a burn are provided. In the methods, a formulation including a Granzyme B inhibitor compound is administered to a subject in need thereof.

In a further aspect, the invention provides methods for healing a burn wound. In the methods, a formulation including a Granzyme B inhibitor compound is administered to the burn wound.

In another aspect, the invention provides methods for reducing or preventing the expansion of the zone of stasis in a burn wound. In the methods, a formulation including a Granzyme B inhibitor compound is administered to the burn wound.

In a further aspect of the invention, the invention provides methods for intradermal delivery of a Granzyme B inhibitor. In the methods, a formulation including a Granzyme B inhibitor compound is administered to the skin.

In the above methods, the formulation can be a gel or solution containing the Granzyme B inhibitor. The gels can be topically administered and the solutions can be administered topically or by injection.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1A is the histology images of Granzyme B (GzmB) staining in normal human skin and burn wound in human, demonstrating the elevated level of GzmB in burn wound. Left scale bar is 100 μm, the right scale bar is 200 μm.

FIG. 1B compares the number of GzmB expressing cells in normal skin and burn wounds in humans.

FIG. 2A compares images of wounds over time (Day 0 to Day 30) after subcutaneous injection of saline (control) and a representative formulation of the invention that includes Compound A demonstrating attenuation of initial burn expansion in the zone of stasis and improved burn wound healing for the representative formulation. The injectable formulation is a solution of Compound A in phosphate buffered saline (0.56 mg/ml).

FIG. 2B is a graphic illustration of the comparative wound healing shown in FIG. 2A (percent of original wound size as a function of days post-wounding) (unpaired student t-test).

FIG. 2C compares wound healing at Day 3 of the comparative wound healing shown in FIG. 2A (percent of original wound size for control (saline) and the representative formulation of the invention that includes Compound A (PBS)) (unpaired student t-test).

FIG. 2D compares wound healing at Day 27 of the comparative wound healing shown in FIG. 2A (percent of original wound size for control (saline) and the representative formulation of the invention that includes Compound A (PBS)) (unpaired student t-test).

FIG. 2E compares falling of scabs (days) in the comparative wound healing shown in FIG. 2A (falling of scabs (days) for control (saline) and the representative formulation of the invention that includes Compound A (PBS)).

FIG. 3A is an image of skin to which a volume (50 μL) of a representative formulation of the invention that includes Compound A was applied. The applied formulation (gel) includes Compound A at a concentration of 0.35% w/v in a vehicle containing propylene glycol (20% w/w), Carbopol 940 (0.5% w/v), methyl paraben (0.2% w/w), propyl paraben (0.02% w/w) and acetate buffer pH 5 (QS). Triethylamine was used to adjust the final formulation pH to 5-6.

FIG. 3B compares concentration of Compound A (μg/μL) in the skin at 4, 6, and 24 hours after application of a volume (50 μL) of a representative formulation of the invention that includes Compound A as described above for FIG. 3A.

FIG. 4A compares images of wounds over time (Day 0 to Day 27) after subcutaneous injection of saline (control) and topical application of a representative formulation of the invention that includes Compound A (gel) demonstrating attenuation of initial burn expansion in the zone of stasis and improved burn wound healing for the representative formulation.

FIG. 4B is a graphic illustration of the comparative wound healing shown in FIG. 4A (percent of original wound size as a function of days post-wounding) (unpaired student t-test).

FIG. 4C compares wound healing at Day 3 of the comparative wound healing shown in FIG. 4A (percent of original wound size for control (saline) and the representative formulation of the invention that includes Compound A (gel)).

FIG. 4D compares wound healing at Day 27 of the comparative wound healing shown in FIG. 4A (percent of original wound size for control (saline) and the representative formulation of the invention that includes Compound A (gel)).

FIG. 4E compares falling of scabs (days) in the comparative wound healing shown in FIG. 4A (falling of scabs (days) for control (saline) and the representative formulation of the invention that includes Compound A (gel)).

FIG. 5A compares epidermal thickness of the healed wound shown in FIG. 2B and FIG. 4B demonstrating that Compound A (gel) improves quality of healed wound.

FIG. 5B compares decorin level of the healed wound shown in FIG. 2B and FIG. 4B demonstrating that Compound A (gel) improves quality of healed wound.

FIG. 5C compares collagen level of the healed wound shown in FIG. 2B and FIG. 4B demonstrating that Compound A (gel) improves quality of healed wound.

FIG. 6A compares images of wounds over time (Day 0 to Day 24) after topical application of vehicle (control) and a representative formulation of the invention that includes Compound A (gel) demonstrating attenuation of initial burn expansion in the zone of stasis and improved burn wound healing for the representative formulation.

FIG. 6B is a graphic illustration of the comparative wound healing shown in FIG. 6A (percent of original wound size as a function of days post-wounding) (unpaired student t-test).

FIG. 6C compares wound healing at Day 3 of the comparative wound healing shown in FIG. 6A (percent of original wound size for control (vehicle) and the representative formulation of the invention that includes Compound A (gel)).

FIG. 7 illustrates ex vivo pig skin permeation showing the amount of Compound A1 (formulated at 3 and 13 mg/mL) in tape-stripped skin sample compared to vehicle control (no active).

FIG. 8 illustrates ex vivo pig skin permeation showing the amount of Compound A1 (formulated at 3 and 13 mg/mL in receptor fluid in tape-stripped skin sample compared to vehicle control (no active).

FIG. 9 is a schematic illustration of a representative synthetic pathway for the preparation of representative compounds (P5-P4-P3-P2-P1 starting from P1) useful in the formulations and methods of the invention.

FIG. 10 is a schematic illustration of another representative synthetic pathway for the preparation of representative compounds (P5-P4-P3-P2-P1 starting from P5) useful in the formulations and methods of the invention.

FIG. 11 is a schematic illustration of a further representative synthetic pathway for the preparation of representative compounds (P5-P4-P3-P2-P1 starting from a component other than P1 or P5) useful in the formulations and methods of the invention.

### DETAILED DESCRIPTION OF THE INVENTION

The present invention provides formulations and methods for the treatment of burns and for burn wound healing. The formulations include a Granzyme B inhibitor compound. In the methods of the invention, the formulation is administered topically or by subcutaneous injection.

Granzyme B is a pro-apoptotic serine protease found in the granules of cytotoxic lymphocytes (CTL) and natural killer (NK) cells. Granzyme B is released towards target cells, along with the pore-forming protein, perforin, resulting in its perforin-dependent internalization into the cytoplasm and subsequent induction of apoptosis (see, for e.g., Medema et al., Eur. J. Immunol. 27:3492-3498, 1997). However, during aging, inflammation, and chronic disease, Granzyme B can also be expressed and secreted by other types of immune (e.g., mast cell, macrophage, neutrophil, and dendritic cells) or non-immune (keratinocyte, chondrocyte) cells and has been shown to possess extracellular matrix remodeling activity (Choy et al., Arterioscler. Thromb. Vasc. Biol. 24(12):2245-2250, 2004 and Buzza et al., J. Biol. Chem. 280:23549-23558, 2005).

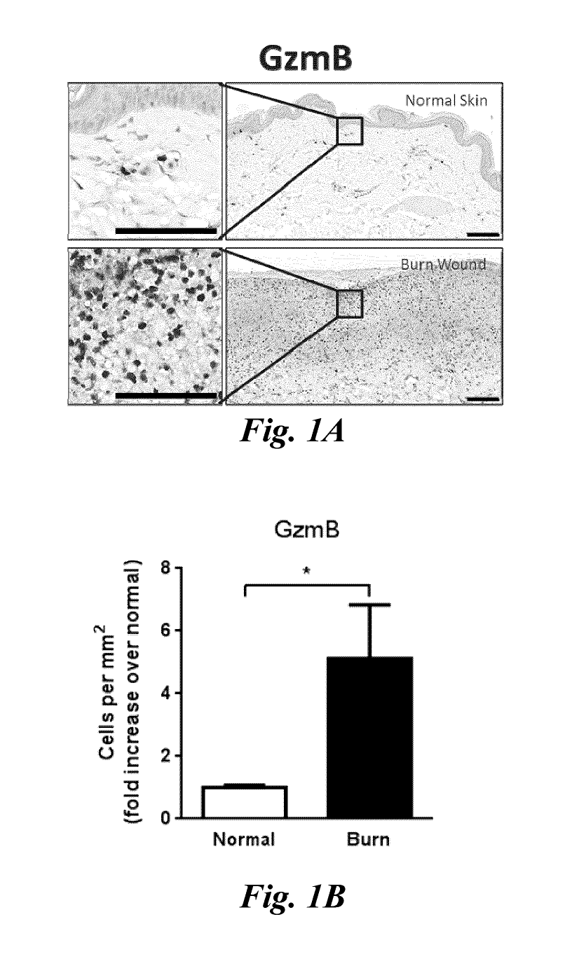

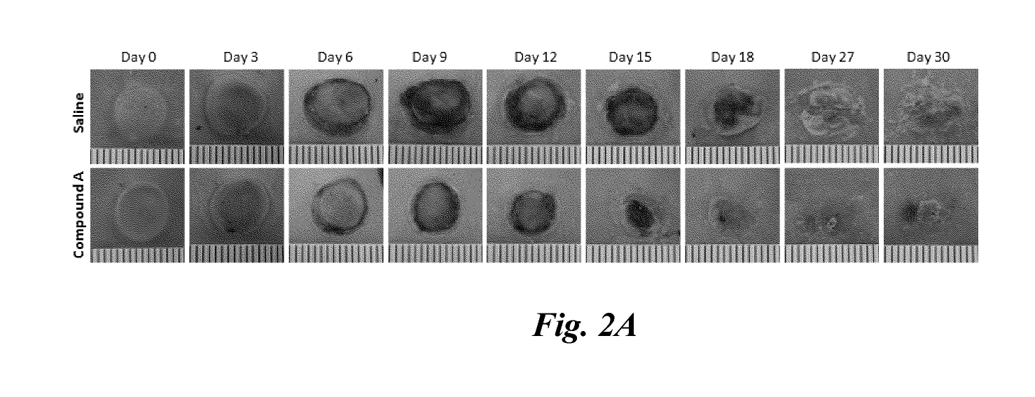

In fact, histology images of Granzyme B (GzmB) staining in normal human skin and burn wound in human demonstrate the elevated level of GzmB in burn wound. FIG. 1A compares histological images for normal and burn wound human skin. The insets clearly show increased GrB levels for burn wound. FIG. 1B compares the number of GzmB expressing cells in normal skin and burn wounds in humans.

Based on the surprising results described herein, and without being bound to theory, it is believed that the inhibition of Granzyme B in tissues at burn wound sites advantageously promotes burn wound healing and therefore improves burn treatment.

In one aspect, the invention provides formulations for treating burn wounds. The formulation includes a Granzyme B inhibitor compound or a pharmaceutically acceptable salt thereof in combination with a pharmaceutically acceptable carrier, and optionally other wound healing ingredients.

In the practice of the invention, it has been advantageously found that the formulations of the invention are effective in penetration of the stratum corneum without significant complete skin penetration. The formulations of the invention are effective for intradermal delivery of the Granzyme B inhibitor compound rather than transdermal delivery, typically a desirable characteristic for systemic administration of therapeutic agents. The effective intradermal delivery result is unexpected as penetration enhancers are used to transport therapeutic agents through the skin rather than to the skin. Without being bound to theory, the advantageous intradermal delivery of the Granzyme B inhibitor compound may be attributed to the nature of the Granzyme B inhibitor compound. Thus, in certain aspects, the invention provides methods for intradermal delivery of a Granzyme B inhibitor.

The Granzyme B inhibitor compound-containing formulations of the invention are effective in burn wound healing and the results demonstrate that these formulations can reduce or prevent the expansion of the zone of stasis and, consequently, work to significantly lessen wound severity. Thus, in certain aspects, the invention provides methods for reducing or preventing the expansion of the zone of stasis in a burn wound.

Granzyme B Inhibitor Compounds

The formulations and methods of the invention use Granzyme B inhibitor compounds having Formula (I):

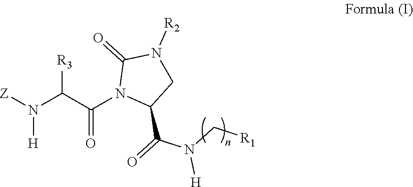

stereoisomers, tautomers, or pharmaceutically acceptable salts thereof, wherein:

R₁ is a heteroaryl group selected from

(a) 1,2,3-triazolyl, and

(b) 1,2,3,4-tetrazolyl;

n is 1 or 2;

R₂ is selected from hydrogen, C1-C6 alkyl, and C3-C6 cycloalkyl;

R₃ is selected from

(a) hydrogen,

(b) C₁-C₄ alkyl optionally substituted with a carboxylic acid, carboxylate, or carboxylate C₁-C₈ ester group (—CO₂H, —CO₂⁻, —C(═O)OC₁-C₈), an amide optionally substituted with an alkylheteroaryl group, or a heteroaryl group;

Z is an acyl group selected from the group

(a)

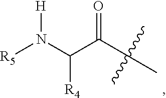

and

(b)

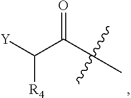

wherein

Y is hydrogen, heterocycle, —NH₂, or C₁-C₄ alkyl;

R₄ is selected from

(i) C₁-C₁₂ alkyl,

(ii) C₁-C₆ heteroalkyl optionally substituted with C₁-C₆ alkyl,

(iii) C₃-C₆ cycloalkyl,

(iv) C₆-C₁₀ aryl,

(v) heterocyclyl,

(vi) C₃-C₁₀ heteroaryl,

(vii) aralkyl, and

(viii) heteroalkylaryl;

R₅ is heteroaryl or —C(═O)—R₁₀,

wherein R₁₀ is selected from

(i) C₁-C₁₂ alkyl optionally substituted with C₆-C₁₀ aryl, C₁-C₁₀ heteroaryl, amino, or carboxylic acid,

(ii) C₁-C₁₀ heteroalkyl optionally substituted with C₁-C₆ alkyl or carboxylic acid,

(iii) C₃-C₆ cycloalkyl optionally substituted with C₁-C₆ alkyl, optionally substituted C₆-C₁₀ aryl, optionally substituted C₃-C₁₀ heteroaryl, amino, or carboxylic acid,

(iv) C₆-C₁₀ aryl optionally substituted with C₁-C₆ alkyl, optionally substituted C₆-C₁₀ aryl, optionally substituted C₃-C₁₀ heteroaryl, amino, or carboxylic acid,

(v) heterocyclyl,

(vi) C₃-C₁₀ heteroaryl,

(vii) aralkyl, and

(viii) heteroalkylaryl.

In certain embodiment, the compounds useful in the formulations and methods of the invention include compounds having Formula (I), stereoisomers, tautomers, or pharmaceutically acceptable salts thereof, wherein:

R₁ is a heteroaryl group selected from

(a) 1,2,3-triazolyl, and

(b) 1,2,3,4-tetrazolyl;

n is 1;

R₂ is selected from hydrogen, C1-C6 alkyl, and C3-C6 cycloalkyl;

R₃ is selected from

(a) hydrogen,

(b) C₁-C₄ alkyl optionally substituted with a carboxylic acid, carboxylate, or carboxylate C₁-C₈ ester group (—CO₂H, —CO₂, —C(═O)OC₁-C₈), an amide optionally substituted with an alkylheteroaryl group, or a heteroaryl group;

Z is an acyl group selected from the group

(a)

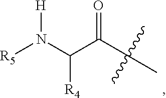

and

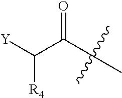

(b)

wherein R₄, R₅, and Y are as described above.

In further embodiments, the compounds useful in the formulations and methods of the invention include compounds having Formula (I), stereoisomers, tautomers, or pharmaceutically acceptable salts thereof, wherein:

R₁ is tetrazole or triazole; n is 1; R₂ is selected from hydrogen, C1-C6 alkyl, and C3-C6 cycloalkyl; R₃ is selected from hydrogen, C₁-C₄ alkyl substituted with a carboxylic acid or carboxylate group, C₁-C₄ alkyl substituted with an amide optionally substituted with an alkylheteroaryl group, or a heteroaryl group; and Z is

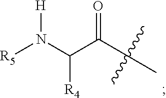

and

R₁ is tetrazole or triazole; n is 1; R₂ is selected from hydrogen, C1-C6 alkyl, and C3-C6 cycloalkyl; R₃ is independently hydrogen, or C₁-C₄ alkyl substituted with a carboxylic acid or carboxylate group, an amide optionally substituted with an alkylheteroaryl group, or a heteroaryl group; and Z is

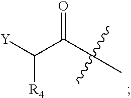

wherein

R₄ is selected from

(i) C₁-C₁₂ alkyl,

(ii) C₃-C₆ cycloalkyl,

(iii) C₆-C₁₀ aryl, and

(iv) C₃-C₁₀ heteroaryl;

R₅ is —C(═O)—R₁₀, wherein R₁₀ is selected from

(i) C₁-C₁₂ alkyl optionally substituted with C₆-C₁₀ aryl, C₁-C₁₀ heteroaryl, amino, or carboxylic acid,

(ii) C₁-C₁₀ heteroalkyl optionally substituted with C₁-C₆ alkyl or carboxylic acid,

(iii) C₃-C₆ cycloalkyl optionally substituted with C₁-C₆ alkyl, optionally substituted C₆-C₁₀ aryl, optionally substituted C₃-C₁₀ heteroaryl, amino, or carboxylic acid,

(iv) C₆-C₁₀ aryl optionally substituted with C₁-C₆ alkyl, optionally substituted C₆-C₁₀ aryl, optionally substituted C₃-C₁₀ heteroaryl, amino, or carboxylic acid,

(v) C₃-C₁₀ heteroaryl; and

Y is hydrogen, C₁-C₄ alkyl, or —NH₂.

In another embodiment, the compounds useful in the formulations and methods of the invention include compounds having Formula (II):

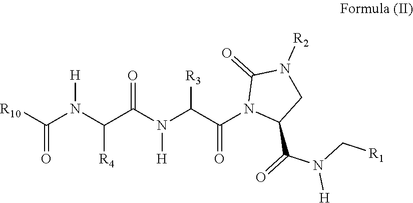

stereoisomers, tautomers, or pharmaceutically acceptable salts thereof, wherein:

R₁, R₂, R₃, R₄, and R₁₀ are as above for Formula (I).

In certain embodiments, R₁₀, when defined as C₁-C₁₂ alkyl substituted with a carboxylic acid or carboxylate group, is:

—(CH₂)ₙ—CO₂H, where n is 2, 3, 4, 5, or 6;

optionally wherein one or more single methylene carbons are substituted with a fluoro, hydroxy, amino, C₁-C₃ alkyl (e.g., methyl), or C₆-C₁₀ aryl group;

optionally wherein one or more single methylene carbons are substituted with two fluoro (e.g., difluoro, perfluoro) or C₁-C₃ alkyl (e.g., gem-dimethyl) groups;

optionally wherein one or more single methylene carbons are substituted with two alkyl groups that taken together with the carbon to which they are attached form a 3, 4, 5, or 6-membered carbocyclic ring (e.g., Spiro groups such as cyclopropyl, cyclobutyl, cyclopentyl, and cyclohexyl); and

optionally wherein adjacent carbon atoms from an unsaturated carbon-carbon bond (e.g., alkenyl such as —CH═CH—) or taken form a benzene ring (e.g., 1,2-, 1,3-, and 1,4-phenylene); or

wherein R₁₀, when defined as C₃-C₆ cycloalkyl substituted with a carboxylic acid or carboxylate group, is:

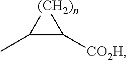

wherein n is 1, 2, 3, or 4; and optionally, for n=3 or 4, wherein adjacent carbon atoms from an unsaturated carbon-carbon bond (e.g., cyclopentenyl or cyclohexenyl).

In certain embodiments, the compounds useful in the formulations and methods of the invention include compounds having Formula (II), stereoisomers, tautomers, or pharmaceutically acceptable salts thereof, wherein:

R₁ is tetrazole or triazole;

R₂ is selected from hydrogen, C1-C6 alkyl, and C3-C6 cycloalkyl;

R₃ is hydrogen, C₁-C₄ alkyl optionally substituted with a carboxylic acid, carboxylate, or a carboxylate ester group; or C₁-C₄ alkyl optionally substituted with an amide, which may be optionally substituted with an alkylheteroaryl group;

R₄ is C₁-C₁₂ alkyl, C₃-C₆ cycloalkyl, C₆-C₁₀ aryl, C₃-C₁₀ heteroaryl, or heterocyclyl; and

R₁₀ is C₁-C₁₂ alkyl optionally substituted with C₆-C₁₀ aryl, C₁-C₁₀ heteroaryl, amino, or carboxylic acid.

In further embodiments, the compounds useful in the formulations and methods of the invention include compounds having Formula (II), stereoisomers, tautomers, or pharmaceutically acceptable salts thereof, wherein:

R₁ is tetrazole or triazole;

R₂ is selected from hydrogen, C1-C6 alkyl, and C3-C6 cycloalkyl; R₃ is hydrogen, C₁-C₄ alkyl optionally substituted with a carboxylic acid, carboxylate, or a carboxylate ester group;

R₄ is C₁-C₈ alkyl or C₃-C₆ cycloalkyl; and

R₁₀ is selected from:

(a) C1-C3 alkyl substituted with C₆-C₁₀ aryl (e.g., phenyl) or C₁-C₁₀ heteroaryl (e.g., triazolyl or tetrazolyl);

(b) —(CH₂)ₙ—CO₂H, where n is 2, 3, 4, 5, or 6;

(c)

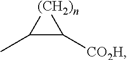

wherein n is 1, 2, 3, or 4.

In one embodiment, the compounds useful in the formulations and methods of the invention include compounds having Formula (II), stereoisomers, tautomers, or pharmaceutically acceptable salts thereof, wherein:

R₁ is tetrazole;

R₂ is selected from hydrogen, C₁-C₆ alkyl (e.g., methyl), and C3-C6 cycloalkyl (e.g., cyclohexyl);

R₃ is hydrogen or C₁-C₄ alkyl optionally substituted with a carboxylic acid, carboxylate, or a carboxylate ester group (e.g., C₂ alkyl substituted with a carboxylic acid, carboxylate, or a carboxylate ester group);

R₄ is C₁-C₈ alkyl (e.g., C₄ alkyl); and

R₁₀ is —(CH₂)ₙ—CO₂H, where n is 2, 3, 4, 5, or 6 (e.g., —(CH₂)ₙ—CO₂H, where n is 2).

Representative compounds of Formula (II) include C1-C5.

In a further embodiment, the compounds useful in the formulations and methods of the invention include compounds having Formula (III):

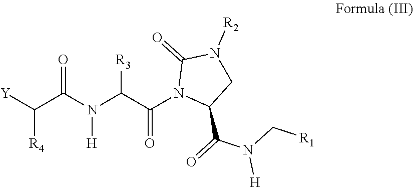

stereoisomers, tautomers, or pharmaceutically acceptable salts thereof, wherein R₁, R₂, R₃, R₄, and Y are as defined above for Formula (I).

In certain embodiments, the compounds useful in the formulations and methods of the invention include compounds having Formula (III), stereoisomers, tautomers, or pharmaceutically acceptable salts thereof, wherein:

R₁ is tetrazole or triazole;

R₂ is selected from hydrogen, C1-C6 alkyl, and C3-C6 cycloalkyl;

R₃ is hydrogen; C₁-C₄ alkyl optionally substituted with a carboxylic acid, carboxylate, or a carboxylate ester group; or C₁-C₄ alkyl optionally substituted with an amide, which may be optionally substituted with an alkylheteroaryl group;

R₄ is C₁-C₁₂ alkyl, C₃-C₆ cycloalkyl, C₆-C₁₀ aryl, C₃-C₁₀ heteroaryl, or heterocyclyl; and

Y is hydrogen, C₁-C₄ alkyl, or —NH₂.

In further embodiments, the compounds useful in the formulations and methods of the invention include compounds having Formula (III), stereoisomers, tautomers, or pharmaceutically acceptable salts thereof, wherein:

R₁ is tetrazole or triazole;

R₂ is selected from hydrogen, C1-C6 alkyl, and C3-C6 cycloalkyl; R₃ is C₁-C₄ alkyl optionally substituted with a carboxylic acid, carboxylate, or a carboxylate ester group;

R₄ is selected from

(i) C₁-C₈ alkyl (e.g., methyl, ethyl, n-propyl, i-propyl),

(ii) C₃-C₆ cycloalkyl (i.e., cyclopropyl, cyclobutyl, cyclopentyl, cyclohexyl),

(iii) C₆-C₁₀ aryl (e.g., phenyl),

(iv) C₃-C₁₀ heteroaryl (e.g., thiophenyl), and

(v) heterocyclyl (e.g., morpholinyl); and

Y is hydrogen.

Representative compounds of Formula (III) include C6.

For the compounds of Formulae (I), (II), or (III), representative substituents R₃ include the following:

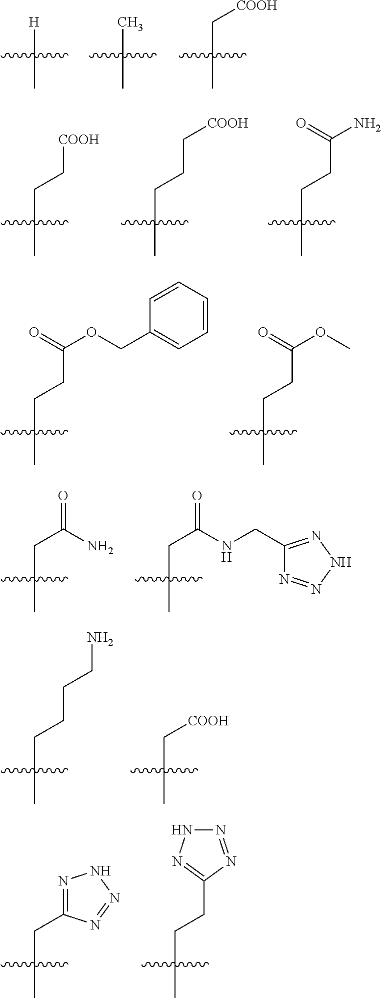

For the compounds of Formulae (I), (II), or (III), representative substituents R₄ include the following:

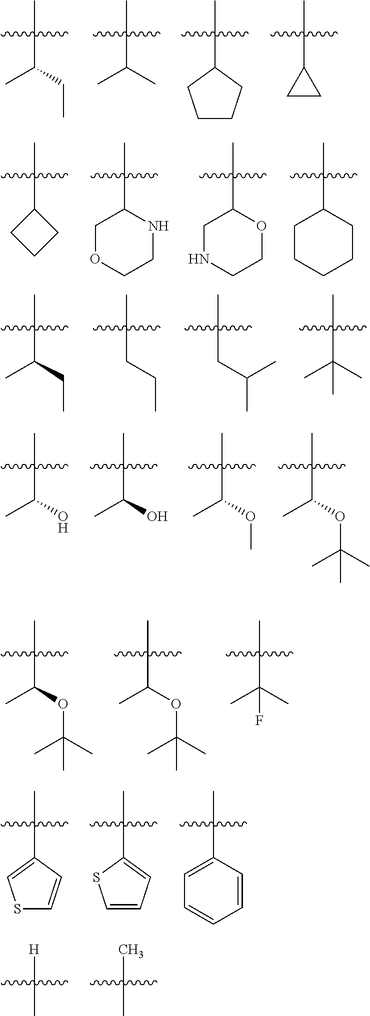

For the compounds of Formulae (I), (II), or (III), representative substituents R₅ include the following:

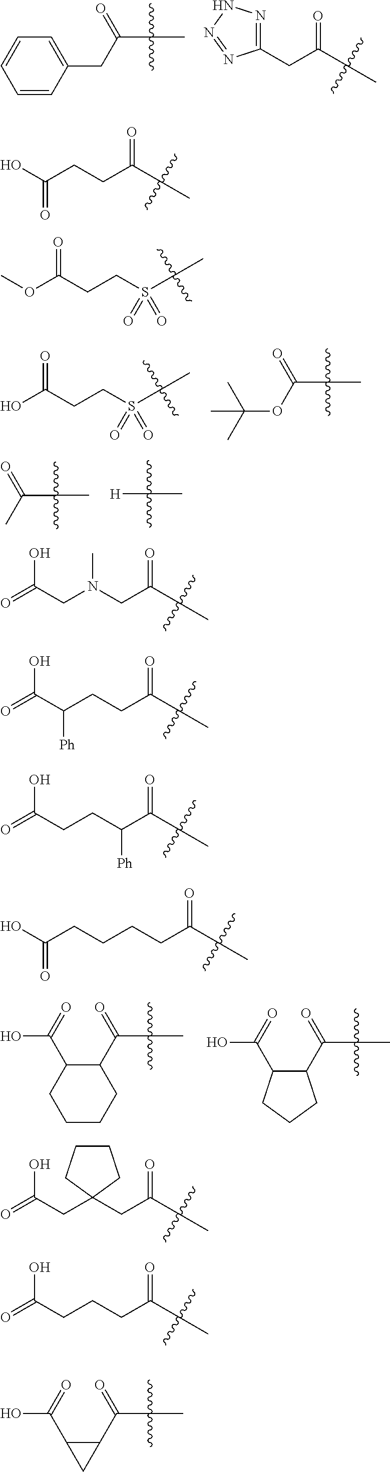

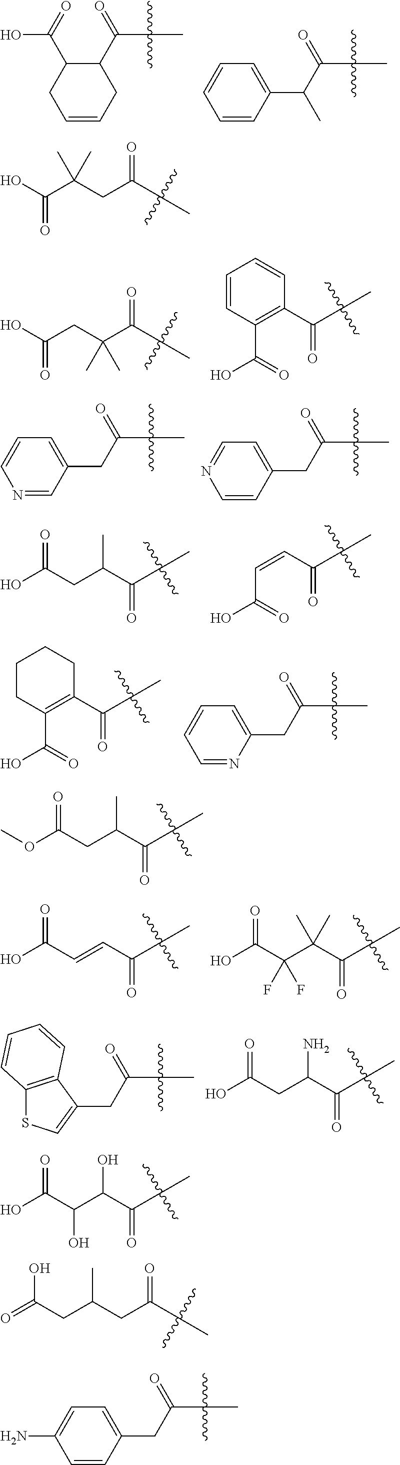

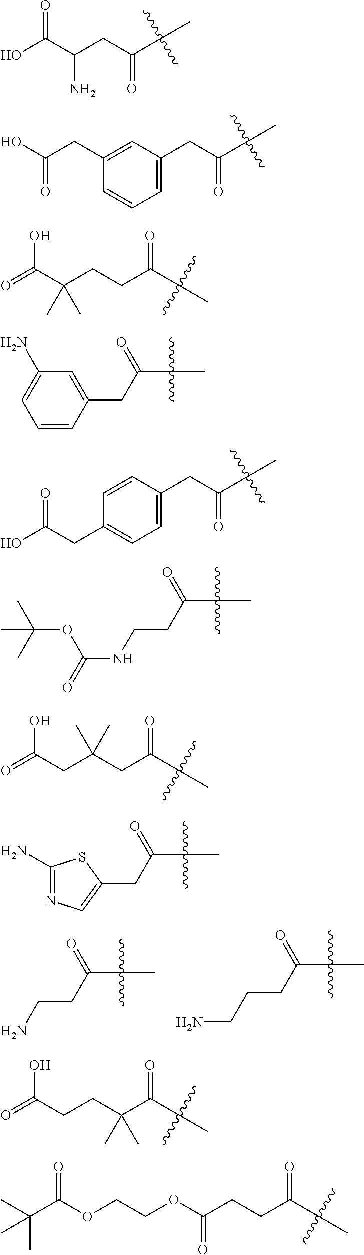

Each of the inhibitor compounds contain asymmetric carbon centers and give rise to stereoisomers (i.e., optical isomers such as diastereomers and enantiomers). It will be appreciated that the present invention includes such diastereomers as well as their racemic and resolved enantiomerically pure forms. It will also be appreciated that in certain configurations, the relative stereochemistry of certain groups may be depicted as “cis” or “trans” when absolute stereochemistry is not shown.

Some of the compounds described herein contain olefinic double bonds, and unless specified otherwise, are meant to include both E and Z geometric isomers.

Certain of the compounds may exist in one or more tautomeric forms (e.g., acid or basic forms depending on pH environment). It will be appreciated that the compounds include their tautomeric forms (i.e., tautomers).

When the compounds are basic, salts may be prepared from pharmaceutically acceptable non-toxic acids, including inorganic and organic acids. Examples of such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, and p-toluenesulfonic acids.

The following definitions unless otherwise indicated.

As used herein, the term “alkyl” refers to a saturated or unsaturated, branched, straight-chain or cyclic monovalent hydrocarbon group derived by the removal of one hydrogen atom from a single carbon atom of a parent alkane, alkene, or alkyne. Representative alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, and prop-2-yn-1-yl; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, and but-3-yn-1-yl; and the like. Where a specific level of saturation is intended, the expressions “alkanyl,” “alkenyl,” and “alkynyl” are used. Alkyl groups include cycloalkyl groups. The term “cycloalkyl” refers to mono-, bi-, and tricyclic alkyl groups having the indicated number of carbon atoms. Representative cycloalkyl groups include cyclopropyl, cyclopentyl, cycloheptyl, adamantyl, cyclododecylmethyl, and 2-ethyl-1-bicyclo[4.4.0]decyl groups. The alkyl group may be unsubstituted or substituted as described below.

“Alkanyl” refers to a saturated branched, straight-chain, or cyclic alkyl group. Representative alkanyl groups include methanyl; ethanyl; propanyls such as propan-1-yl, propan-2-yl(isopropyl), and cyclopropan-1-yl; butanyls such as butan-1-yl, butan-2-yl (sec-butyl), 2-methyl-propan-1-yl(isobutyl), 2-methyl-propan-2-yl(t-butyl), and cyclobutan-1-yl; and the like. The alkanyl group may be substituted or unsubstituted. Representative alkanyl group substituents include

—R₁₄, —OR₁₄, —SR₁₄, —NR₁₄(R₁₅),

—X, —CX₃, —CN, —NO₂,

—C(═O)R₁₄, —C(═O)OR₁₄, —C(═O)NR₁₄(R₁₅), —C(═O)SR₁₄,

—C(═NR₁₄)R₁₄, —C(═NR₁₄)OR₁₄, —C(═NR₁₄)NR₁₄(R₁₅), —C(═NR₁₄)SR₁₄,

—C(═S)R₁₄, —C(═S)OR₁₄, —C(═S)NR₁₄(R₁₅), —C(═S)SR₁₄,

—NR₁₄C(═O)NR₁₄(R₁₅), —NR₁₄(═NR₁₄)NR₁₄(R₁₅), —NR₁₄C(═S)NR₁₄(R₁₅),

—S(═O)₂R₁₄, —S(═O)₂OR₁₄, —S(═O)₂NR₁₄(R₁₅),

—OC(═O)R₁₄, —OC(═O)OR₁₄, —OC(═O)NR₁₄(R₁₅), —OC(═O)SR₁₄,

—OS(═O)₂OR₁₄, —OS(═O)₂NR₁₄(R₁₅), and

—OP(═O)₂(OR₁₄),

wherein each X is independently a halogen; and R₁₄ and R₁₅ are independently hydrogen, C1-C6 alkyl, C6-C14 aryl, arylalkyl, C3-C10 heteroaryl, and heteroarylalkyl, as defined herein.

In certain embodiments, two hydrogen atoms on a single carbon atom can be replaced with ═O, ═NR₁₂, or ═S.

“Alkenyl” refers to an unsaturated branched, straight-chain, cyclic alkyl group, or combinations thereof having at least one carbon-carbon double bond derived by the removal of one hydrogen atom from a single carbon atom of a parent alkene. The group may be in either the cis or trans conformation about the double bond(s). Representative alkenyl groups include ethenyl; propenyls such as prop-1-en-1-yl, prop-1-en-2-yl, prop-2-en-1-yl (allyl), prop-2-en-2-yl, and cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, and cyclobuta-1,3-dien-1-yl; and the like. The alkenyl group may be substituted or unsubstituted. Representative alkenyl group substituents include

—R₁₄,

—X, —CX₃, —CN,

—C(═O)R₁₄, —C(═O)OR₁₄, —C(═O)NR₁₄(R₁₅), —C(═O)SR₁₄,

—C(═NR₁₄)R₁₄, —C(═NR₁₄)OR₁₄, —C(═NR₁₄)NR₁₄(R₁₅), —C(═NR₁₄)SR₁₄,

—C(═S)R₁₄, —C(═S)OR₁₄, —C(═S)NR₁₄(R₁₅), —C(═S)SR₁₄,

wherein each X is independently a halogen; and R₁₄ and R₁₅ are independently hydrogen, C1-C6 alkyl, C6-C14 aryl, arylalkyl, C3-C10 heteroaryl, and heteroarylalkyl, as defined herein.

“Alkynyl” refers to an unsaturated branched, straight-chain, or cyclic alkyl group having at least one carbon-carbon triple bond derived by the removal of one hydrogen atom from a single carbon atom of a parent alkyne. Representative alkynyl groups include ethynyl; propynyls such as prop-1-yn-1-yl and prop-2-yn-1-yl; butynyls such as but-1-yn-1-yl, but-1-yn-3-yl, and but-3-yn-1-yl; and the like. The alkynyl group may be substituted or unsubstituted. Representative alkynyl group substituents include those as described above for alkenyl groups.

The term “haloalkyl” refers to an alkyl group as defined above having the one or more hydrogen atoms replaced by a halogen atom. Representative haloalkyl groups include halomethyl groups such as chloromethyl, fluoromethyl, and trifluoromethyl groups; and haloethyl groups such as chloroethyl, fluoroethyl, and perfluoroethyl groups. The term “heteroalkyl” refers to an alkyl group having the indicated number of carbon atoms and where one or more of the carbon atoms is replaced with a heteroatom selected from O, N, or S. Where a specific level of saturation is intended, the expressions “heteroalkanyl,” “heteroalkenyl,” and “heteroalkynyl” are used. Representative heteroalkyl groups include ether, amine, and thioether groups. Heteroalkyl groups include heterocyclyl groups. The term “heterocyclyl” refers to a 5- to 10-membered non-aromatic mono- or bicyclic ring containing 1-4 heteroatoms selected from 0, S, and N. Representative heterocyclyl groups include pyrrolidinyl, piperidinyl, piperazinyl, tetrahydrofuranyl, tetrahydropuranyl, and morpholinyl groups. The heteroalkyl group may be substituted or unsubstituted. Representative heteroalkyl substituents include

—R₁₄, —OR₁₄, —SR₁₄, —NR₁₄(R₁₅),

—X, —CX₃, —CN, —NO₂,

—C(═O)R₁₄, —C(═O)OR₁₄, —C(═O)NR₁₄(R₁₅), —C(═O)SR₁₄,

—C(═NR₁₄)R₁₄, —C(═NR₁₄)OR₁₄, —C(═NR₁₄)NR₁₄(R₁₅), —C(═NR₁₄)SR₁₄,

—C(═S)R₁₄, —C(═S)OR₁₄, —C(═S)NR₁₄(R₁₅), —C(═S)SR₁₄,

—NR₁₄C(═O)NR₁₄(R₁₅), —NR₁₄(═NR₁₄)NR₁₄(R₁₅), —NR₁₄C(═S)NR₁₄(R₁₅),

—S(═O)₂R₁₄, —S(═O)₂OR₁₄, —S(═O)₂NR₁₄(R₁₅),

—OC(═O)R₁₄, —OC(═O)OR₁₄, —OC(═O)NR₁₄(R₁₅), —OC(═O)SR₁₄,

—OS(═O)₂OR₁₄, —OS(═O)₂NR₁₄(R₁₅), and

—OP(═O)₂(OR₁₄),

wherein each X is independently a halogen; and R₁₄ and R₁₅ are independently hydrogen, C1-C6 alkyl, C6-C14 aryl, arylalkyl, C3-C10 heteroaryl, and heteroarylalkyl, as defined herein.

In certain embodiments, two hydrogen atoms on a single carbon atom can be replaced with ═O, ═NR₁₂, or ═S.

The term “alkoxy” refers to an alkyl group as described herein bonded to an oxygen atom. Representative C1-C3 alkoxy groups include methoxy, ethoxy, propoxy, and isopropoxy groups.

The term “alkylamino” refers an alkyl group as described herein bonded to a nitrogen atom. The term “alkylamino” includes monoalkyl- and dialkylaminos groups. Representative C1-C6 alkylamino groups include methylamino, dimethylamino, ethylamino, methylethylamino, diethylamino, propylamino, and isopropylamino groups.

The term “alkylthio” refers an alkyl group as described herein bonded to a sulfur atom. Representative C1-C6 alkylthio groups include methylthio, propylthio, and isopropylthio groups.

The term “aryl” refers to a monovalent aromatic hydrocarbon group derived by the removal of one hydrogen atom from a single carbon atom of a parent aromatic ring system. Suitable aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, the aryl group is a C5-C14 aryl group. In other embodiments, the aryl group is a C5-C10 aryl group. The number of carbon atoms specified refers to the number of carbon atoms in the aromatic ring system. Representative aryl groups are phenyl, naphthyl, and cyclopentadienyl. The aryl group may be substituted or unsubstituted. Representative aryl group substituents include

—R₁₄, —OR₁₄, —SR₁₄, —NR₁₄(R₁₅),

—X, —CX₃, —CN, —NO₂,

—C(═O)R₁₄, —C(═O)OR₁₄, —C(═O)NR₁₄(R₁₅), —C(═O)SR₁₄,

—C(═NR₁₄)R₁₄, —C(═NR₁₄)OR₁₄, —C(═NR₁₄)NR₁₄(R₁₅), —C(═NR₁₄)SR₁₄,

—C(═S)R₁₄, —C(═S)OR₁₄, —C(═S)NR₁₄(R₁₅), —C(═S)SR₁₄,

—NR₁₄C(═O)NR₁₄(R₁₅), —NR₁₄(═NR₁₅)NR₁₄(R₁₅), —NR₁₄C(═S)NR₁₄(R₁₅),

—S(═O)₂R₁₄, —S(═O)₂OR₁₄, —S(═O)₂NR₁₄(R₁₅),

—OC(═O)R₁₄, —OC(═O)OR₁₄, —OC(═O)NR₁₄(R₁₅), —OC(═O)SR₁₄,

—OS(═O)₂OR₁₄, —OS(═O)₂NR₁₄(R₁₅), and

—OP(═O)₂(OR₁₄),

wherein each X is independently a halogen; and R₁₄ and R₁₅ are independently hydrogen, C1-C6 alkyl, C6-C14 aryl, arylalkyl, C3-C10 heteroaryl, and heteroarylalkyl, as defined herein.

The term “aralkyl” refers to an alkyl group as defined herein with an aryl group, optionally substituted, as defined herein substituted for one of the alkyl group hydrogen atoms. Suitable aralkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the terms aralkanyl, aralkenyl, and aralkynyl are used. In certain embodiments, the aralkyl group is a C6-C20 aralkyl group, (e.g., the alkanyl, alkenyl, or alkynyl moiety of the aralkyl group is a C1-C6 group and the aryl moiety is a C5-C14 group). In other embodiments, the aralkyl group is a C6-C13 aralkyl group (e.g., the alkanyl, alkenyl, or alkynyl moiety of the aralkyl group is a C1-C3 group and the aryl moiety is a C5-C10 aryl group. In certain embodiments, the aralkyl group is a benzyl group.

The term “heteroaryl” refers to a monovalent heteroaromatic group derived by the removal of one hydrogen atom from a single atom of a parent heteroaromatic ring system, which may be monocyclic or fused ring (i.e., rings that share an adjacent pair of atoms). A “heteroaromatic” group is a 5- to 14-membered aromatic mono- or bicyclic ring containing 1-4 heteroatoms selected from O, S, and N. Representative 5- or 6-membered aromatic monocyclic ring groups include pyridine, pyrimidine, pyridazine, furan, thiophene, thiazole, oxazole, and isooxazole. Representative 9- or 10-membered aromatic bicyclic ring groups include benzofuran, benzothiophene, indole, pyranopyrrole, benzopyran, quionoline, benzocyclohexyl, and naphthyridine. Suitable heteroaryl groups include groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, the heteroaryl group is a 5-14 membered heteroaryl group. In other embodiments, the heteroaryl group is a 5-10 membered heteroaryl group. Preferred heteroaryl groups are those derived from thiophene, pyrrole, benzothiophene, benzofuran, indole, pyridine, quinoline, imidazole, oxazole, and pyrazine. The heteroaryl group may be substituted or unsubstituted. Representative heteroaryl group substituents include those described above for aryl groups.

The term “heteroarylalkyl” refers to an alkyl group as defined herein with a heteroaryl group, optionally substituted, as defined herein substituted for one of the alkyl group hydrogen atoms. Where specific alkyl moieties are intended, the terms heteroarylalkanyl, heteroarylalkenyl, or heteroarylalkynyl are used. In certain embodiments, the heteroarylalkyl group is a 6-20 membered heteroarylalkyl (e.g., the alkanyl, alkenyl or alkynyl moiety of the heteroarylalkyl is a C1-C6 group and the heteroaryl moiety is a 5-14-membered heteroaryl group. In other embodiments, the heteroarylalkyl group is a 6-13 membered heteroarylalkyl (e.g., the alkanyl, alkenyl or alkynyl moiety is C1-C3 group and the heteroaryl moiety is a 5-10-membered heteroaryl group).

The term “acyl” group refers to the —C(═O)—R′ group, where R′ is selected from optionally substituted alkyl, optionally substituted aryl, and optionally substituted heteroaryl, as defined herein.

The term “halogen” or “halo” refers to fluoro, chloro, bromo, and iodo groups.

The term “substituted” refers to a group in which one or more hydrogen atoms are each independently replaced with the same or different substituent(s).

The term “pharmaceutically acceptable salts” refers to salts prepared from pharmaceutically acceptable bases including inorganic bases and organic bases. Representative salts derived from inorganic bases include aluminum, ammonium, calcium, copper, ferric, ferrous, lithium, magnesium, manganic, manganous, ammonium, potassium, sodium, and zinc salts. Representative salts derived from pharmaceutically acceptable organic bases include salts of primary, secondary and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and trimethamine.

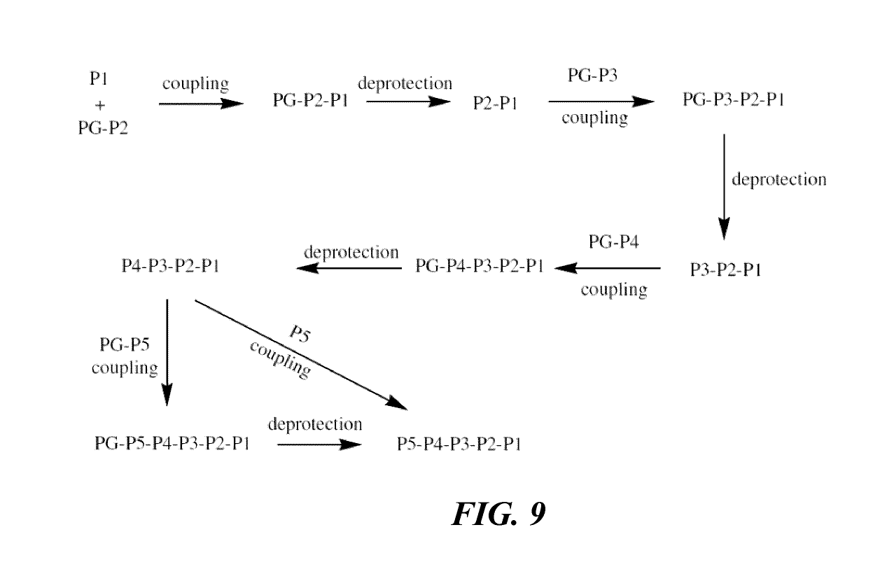

Representative compounds and related intermediates were prepared from commercially available starting materials or starting materials prepared by conventional synthetic methodologies. Representative compounds were prepared according to Methods A to C as described below and illustrated in FIGS. 9-11. The preparations of certain intermediates (I-1 to I-4) useful in the preparation of compounds of the invention are described in the Synthetic Intermediate section below.

FIGS. 9-11 present schematic illustrations of representative synthetic pathways for the preparation of representative compounds of the invention P5-P4-P3-P2-P1. As used herein, “P5-P4-P3-P2-P1” refers to compounds of the invention prepared from five (5) components: P1, P2, P3, P4, and P5. Protected version of the components useful in the preparation of the compounds of the invention are designated as, for example, “PG-P2,” “PG-P2-P1,” “PG-P3,” and “PG-P3-P2-P1,” where “PG” is refers to a protecting group that allows for the coupling of, for example, P1 to P2 or P3 to P1-P2, and that is ultimately removed to provide, for example, P1-P2 or P1-P2-P3.

FIG. 9 is a schematic illustration of another representative synthetic pathway for the preparation of representative compounds of the invention P5-P4-P3-P2-P1 starting from P5. In this pathway, compound P5-P4-P3-P2-P1 is prepared in a stepwise manner starting with P5 by sequential coupling steps, separated as appropriate by deprotection steps and other chemical modifications. As shown in FIG. 9, P5 is coupled with PG-P4 to provide P5-P4-PG, which is then deprotected to provide P5-P4 and ready for coupling with the next component, P3-PG. The process is continued with subsequent couplings PG-P2 with P5-P4-P3 and PG-P1 with P5-P4-P3-P2 to ultimately provide P5-P4-P3-P2-P1.

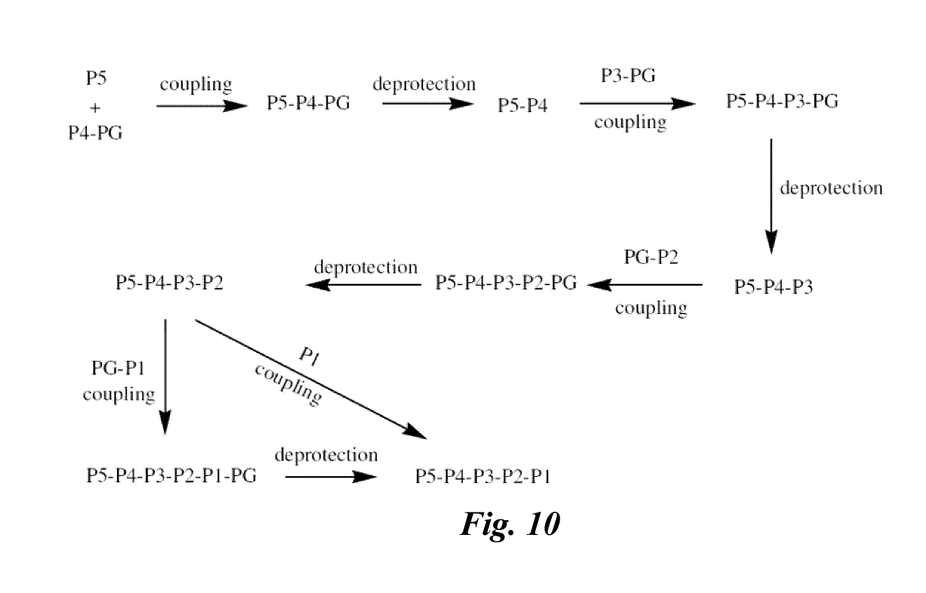

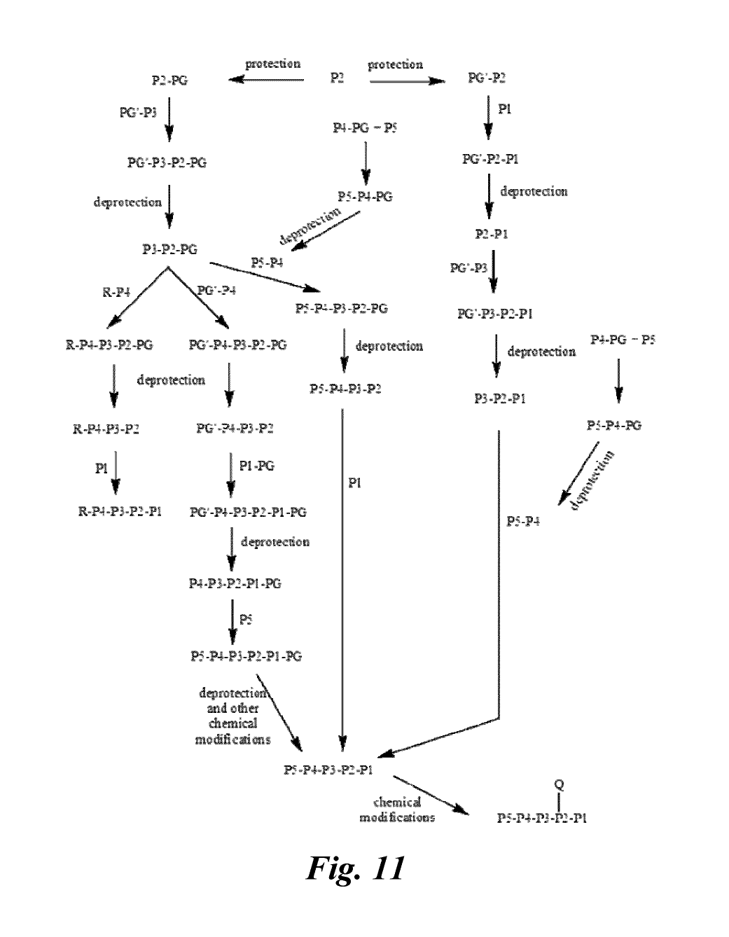

FIG. 10 is a schematic illustration of a representative synthetic pathway for the preparation of representative compounds of the invention P5-P4-P3-P2-P1 starting from P1. In this pathway, compound P5-P4-P3-P2-P1 is prepared in a stepwise manner starting with P1 by sequential coupling steps, separated as appropriate by deprotection steps and other chemical modifications. As shown in FIG. 10, P1 is coupled with PG-P2 to provide PG-P2-P1, which is then deprotected to provide P2-P1 and ready for coupling with the next component, PG-P3. The process is continued with subsequent couplings PG-P4 with P3-P2-P1 and PG-P5 with P4-P3-P2-P1 to ultimately provide P5-P4-P3-P2-P1.

FIG. 11 is a schematic illustration of a further representative synthetic pathway for the preparation of representative compounds of the invention P5-P4-P3-P2-P1 starting from a component other than P1 or P5. In this pathway, compound P5-P4-P3-P2-P1 is prepared in a stepwise manner starting with P2 by sequential coupling steps, separated as appropriate by deprotection steps and other chemical modifications. As shown in FIG. 11, there are multiple pathways to P5-P4-P3-P2-P1. Examples C1-C6 were prepared by this method.

The preparation of representative compounds and their characterization are described in Examples C1-C6. The structures of representative compounds are set forth in Table 1.

TABLE 1
Representative Compounds.

| Cmpd # | Structure |
|---|---|
| C1 | 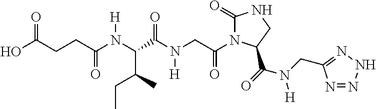 [SMILES: CC[C@H](C)[C@H](NC(=O)CCC(=O)O)C(=O)NCC(=O)N1C(=O)NC[C@H]1C(=O)NCc1nn[nH]n1] |
| C2 | 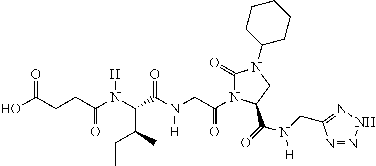 [SMILES: CC[C@H](C)[C@H](NC(=O)CCC(=O)O)C(=O)NCC(=O)N1C(=O)N(C2CCCCC2)C[C@H]1C(=O)NCc1nn[nH]n1] |
| C3 | 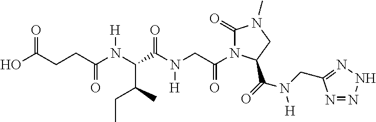 [SMILES: CC[C@H](C)[C@H](NC(=O)CCC(=O)O)C(=O)NCC(=O)N1C(=O)N(C)C[C@H]1C(=O)NCc1nn[nH]n1] |
| C4 | 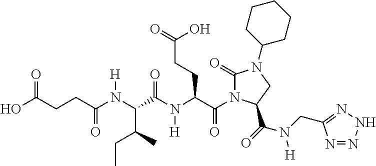 [SMILES: CC[C@H](C)[C@H](NC(=O)CCC(=O)O)C(=O)N[C@@H](CCC(=O)O)C(=O)N1C(=O)N(C2CCCCC2)C[C@H]1C(=O)NCc1nn[nH]n1] |
| C5 | 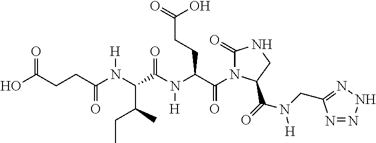 [SMILES: CC[C@H](C)[C@H](NC(=O)CCC(=O)O)C(=O)N[C@@H](CCC(=O)O)C(=O)N1C(=O)NC[C@H]1C(=O)NCc1nn[nH]n1] |
| C6 | 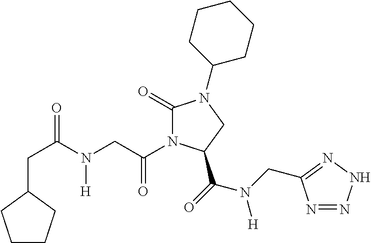 [SMILES: O=C(CC1CCCC1)NCC(=O)N1C(=O)N(C2CCCCC2)C[C@H]1C(=O)NCc1nn[nH]n1] |

A general kinetic enzyme assay useful for determining the inhibitory activity of the representative compounds useful in the formulations and methods of the invention is described in Examples D1 and D4.

A Granzyme B enzymatic inhibition assay is described in Example D2 and Example D5. The compounds identified in Table 1 exhibited Granzyme B inhibitory activity. In certain embodiments, select compounds exhibited IC₅₀<50,000 nM. In other embodiments, select compounds exhibited IC₅₀<10,000 nM. In further embodiments, select compounds exhibited IC₅₀<1,000 nM. In still further embodiments, select compounds exhibited IC₅₀<100 nM. In certain embodiments, select compounds exhibited IC₅₀ from 10 nM to 100 nM, preferably from 1 nM to 10 nM, more preferably from 0.1 nM to 1 nM, and even more preferably from 0.01 nM to 0.1 nM.

A caspase enzymatic inhibition assay is described in Example D3 and Example D6. None of the compounds tested demonstrated an ability to significantly inhibit any of the caspases evaluated at a concentration of 50 μM. In certain embodiments, the compounds exhibited less than 50% inhibition at 50 μM. In other embodiments, the compounds exhibited greater than 50% inhibition at 50 μM, but less than 10% inhibition at 25 μM. The results demonstrate that select compounds selectively inhibit Granzyme B without significantly inhibiting caspases.

A fibronectin cleavage assay is described in Example D7.

Formulations

As noted above, the formulations of the invention include a Granzyme B inhibitor as described herein. A representative Granzyme B inhibitor compound useful in these formulations is 4-(((2S,3S)-1-(((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)amino)-4-oxobutanoic acid (referred to herein Compound A or Compound A1 depending on the preparation method as described in Example C2), and pharmaceutically acceptable salts thereof.

The preparation of Compound A and Compound A1 are described in Example C2.

Performance properties of representative formulations of the invention are described in Example 1 (Compound 1 formulations) and Example 2 (Compound 1A formulations).

In certain embodiments, the Granzyme B inhibitor (e.g., Compound A or Compound A1) is present in the formulation in an amount from about 0.25 to about 25.0 mg/mL of the formulation. In certain embodiments, the Granzyme B inhibitor is present in an amount from about 3.0 to about 15 mg/mL of the formulation. In other embodiments, the Granzyme B inhibitor is present in an amount from about 10.0 to about 15.0 mg/mL of the formulation. In one embodiment, the Granzyme B inhibitor is present in about 10.0 mg/mL of the formulation.

The pH of the formulations of the invention can be readily varied as desired by adjustment with, for example, a base such a triethanol amine. In certain embodiments, the formulation pH is from about 4.0 to about 7.4. In other embodiments, the formulation pH is from about 4.0 to about 6.5. In other embodiments, such as for topical application to the skin, the formulation pH is about 6.0.

In certain embodiments, the formulations of the invention are aqueous formulations that also include organic components. The aqueous formulations are buffered and have a pH in the range from about 4 to about 7, including from about 4 to 5, 4 to 7, and 5 to 7. In certain embodiments, the pH is from about 4.0 to about 6.5. In other embodiments, the pH is about 6.0. Suitable buffers include those useful for pharmaceutical and cosmetic compositions that are topically administered or administered by injection. Representative buffers include acetate and phosphate buffers.

In the practice of the invention, it was determined that the Granzyme B inhibitor skin permeability decreases when the pH of the formulation increases.

In addition to the Granzyme B inhibitor compound, the formulations of the invention include one or more penetration enhancers. Suitable penetration enhancers include propylene glycol (PG), urea, Tween 80, dimethyl isosorbide (DMI), Transcutol, N-methyl-2-pyrollidone (MNP). The amount of penetration enhancer can be carried to achieve the desired formulation properties.

A representative penetration enhancer is propylene glycol (PG). The amount of propylene glycol present in the formulations can range from about 5 to 80 percent by weight based on the total weight of the formulation. In certain embodiments, propylene glycol is present in an amount from about 15 to about 25 percent by weight based on the total weight of the formulation. In other embodiments, propylene glycol is present in an amount about 20 percent by weight based on the total weight of the formulation. For certain topical applications, propylene glycol can be used in an amount up to about 80% w/w.

It will be appreciated that suitable polyols other than propylene glycol can be used in the formulations. Propylene glycol or other suitable polyols provide for hydrogel formulation and prevent rapid drying of the gel. Compared to other polyols, such as glycerin, propylene glycol offers the advantage of being a penetration enhancer and also a better solvent or co-solvent.

Representative formulations of the invention include a Granzyme B inhibitor (0.5 to 15 mg/mL), penetration enhancer (propylene glycol, 15 to 25 percent by weight), and aqueous acetate buffer at pH 5.

In certain embodiments, the formulation further includes one or more viscosity enhancers or gelling agents. Suitable viscosity enhancers include Carbopols, Carbomers, carboxymethyl cellulose (CMC), starches, vegetable gums, and sugars.

Representative viscosity enhancers include crosslinked polyacrylate polymers, such as polyacrylate polymers crosslinked with ethers of pentaerythritol (e.g., Carbopol 940). The viscosity enhancer is typically present in the formulations in an amount from about 0.1 to about 5.0 percent by weight based on the total weight of the formulation (e.g., 0.5 percent by weight based on the total weight of the formulation). When Carbopol 940 is used, formulations containing less than about 0.5% w/w are lotions rather than gels, and at pH less than about 5, the formulation is not viscous.

For formulations that include Carbopol 940, which is pH sensitive, the pH is from about 5 and about 6 to obtain the formulation as a gel. The final pH of the formulation can be adjusted to achieve the desired pH range by using a suitable base for pharmaceutically acceptable base (e.g., sodium hydroxide, triethylamine, or triethanolamine). In certain embodiments, the pH of the formulation is adjusted with triethanolamine.

It has been observed that greater concentrations of Granzyme B inhibitor in gel formulations is achieved with increased viscosity enhancer (e.g., Carbopol 940) concentration. In certain embodiments, the viscosity enhancer (e.g., Carbopol 940) concentration is from about 0.5 to 2 percent by weight of the formulation (e.g. a gel formulation that includes about 10 mg/mL Granzyme B inhibitor). For a gel formulation that includes, for example, 20 mg/mL Granzyme B inhibitor, the viscosity enhancer (e.g., Carbopol 940) concentration is up to about 5 percent by weight of the formulation.

Representative formulations of the invention include a Granzyme B inhibitor (0.5 to 15 mg/mL), penetration enhancer (propylene glycol, 15 to 25 percent by weight), viscosity enhancer (Carbopol 940, 0.5 to 5 percent by weight), and aqueous acetate buffer at pH 5 (titrated to pH 6.0 with triethanolamine).

In certain embodiments, the formulation further includes one or more preservatives. Suitable preservatives include benzoic acid, EDTA, benzalkonium chloride, and parabens.

Representative parabens include methyl paraben and propyl paraben (e.g., methyl paraben at about 0.2 and propyl paraben at about 0.02 percent by weight based on the total weight of the formulation).

In one embodiment, the formulation for topical administration is a gel that includes the Granzyme B inhibitor (e.g., Compound A or A1) at a concentration of 0.35% w/v in a vehicle containing propylene glycol (20% w/w), Carbopol 940 (0.5% w/v), methyl paraben (0.2% w/w), propyl paraben (0.02% w/w) and acetate buffer pH 5 (QS), adjusted to the final formulation pH of 5-6 with triethylamine.

In another embodiment, the formulation for topical administration is a gel that includes the Granzyme B inhibitor (e.g., Compound A or A1) at a concentration of 10 mg/mL in a vehicle containing propylene glycol (20% w/w), Carbopol 940 (0.5 to 2.0% w/v), methyl paraben (0.2% w/w), propyl paraben (0.02% w/w), and acetate buffer pH 5 (QS), adjusted to the final formulation pH of 6 with triethanolamine.

In one embodiment, the formulation is an injectable formulation that includes a Granzyme B inhibitor at a concentration of 0.25 to 25 mg/mL in a pharmaceutically acceptable injection vehicle (e.g., PBS).

The formulations of the invention can further include one or more carriers acceptable for the mode of administration of the preparation, be it by topical administration, lavage, epidermal administration, sub-epidermal administration, dermal administration, subdermal administration, transdermal administration, subcutaneous administration, injection, or any other mode suitable for the selected treatment. Topical administration includes administration to external body surfaces (e.g., skin) as well as to internal body surfaces (e.g., mucus membranes). Suitable carriers are those known in the art for use in such modes of administration.

Suitable compositions can be formulated by means known in the art and their mode of administration and dose determined by a person of skill in the art. For example, Compound A can be dissolved in sterile water or saline or a pharmaceutically acceptable vehicle used for administration of non-water soluble compounds. Many suitable formulations are known including ointments, pastes, gels, hydrogels, foams, creams, powders, lotions, oils, semi-solids, soaps, medicated soaps, shampoos, medicated shampoos, sprays, films, or solutions which can be used topically or locally to administer a compound. Many techniques known to one of skill in the art are described in Remington: the Science & Practice of Pharmacy by Alfonso Gennaro, 20th ed., Williams & Wilkins, (2000).

The formulations can further include excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers.

The formulations of the invention or for use in the methods disclosed herein can be administered in combination with one or more other therapeutic agents as appropriate. Compound A or pharmaceutical compositions in accordance with this invention or for use in the methods disclosed herein can be administered by means of a medical device or appliance such as an implant or wound dressing. Also, implants can be devised that are intended to contain and release such compounds or compositions. An example would be an implant made of a polymeric material adapted to release the compound over a period of time.

In certain embodiments, the formulations of the invention “comprise” the described components and may include other components. In other embodiments, the formulations of the invention “consist essentially of” the described components and may include other components that do not materially affect the characteristic properties of the formulation. In other embodiments, the formulations of the invention “consist of” the described components and do not include other components.

The properties and burn wound healing effectiveness of representative formulations of the invention are described below.

In another aspect, the invention provides methods for treating burns, healing burn wounds, reducing or preventing the expansion of the zone of stasis in a burn wound, and intradermal delivery of a Granzyme B inhibitor.

In certain embodiments, the methods comprise administering a therapeutically effective amount of a Granzyme B inhibitor or a formulation that includes a Granzyme B inhibitor to a subject in need thereof. Representative routes of administration include topical administration and administration by injection.

A “therapeutically effective amount” refers to an amount effective, at dosages and for periods of time necessary, to achieve the desired therapeutic result, such as burn wound healing or reduced levels of Granzyme B activity. A therapeutically effective amount of a compound may vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the Granzyme B inhibitor are outweighed by the therapeutically beneficial effects.

It is to be noted that dosage values can vary with the severity of the condition to be alleviated. For any particular subject, specific dosage regimens can be adjusted over time according to the individual need and the professional judgment of the person administering or supervising the administration of the compositions. Dosage ranges set forth herein are exemplary only and do not limit the dosage ranges that can be selected by a medical practitioner. The amount of active compound in the composition can vary according to factors such as the disease state, age, sex, and weight of the subject. Dosage regimens can be adjusted to provide the optimum therapeutic response. For example, a single bolus can be administered, several divided doses can be administered over time or the dose can be proportionally reduced or increased as indicated by the exigencies of the therapeutic situation.

In the methods, the administration of Granzyme B inhibitor can be a local administration (e.g., administration to the site), or a topical administration to a site (e.g., wound).

The term “subject” or “patient” is intended to include mammalian organisms. Examples of subjects or patients include humans and non-human mammals, e.g., nonhuman primates, dogs, cows, horses, pigs, sheep, goats, cats, mice, rabbits, rats, and transgenic non-human animals. In specific embodiments of the invention, the subject is a human.

The term “administering” includes any method of delivery of Granzyme B inhibitor or a pharmaceutical composition comprising Granzyme B inhibitor into a subject's system or to a particular region in or on a subject.

As used herein, the term “applying” refers to administration of the Granzyme B inhibitor that includes spreading, covering (at least in part), or laying on of the compound.

As used herein, the terms “treating” or “treatment” refer to a beneficial or desired result including, but not limited to, alleviation or amelioration of one or more symptoms, diminishing the extent of a disorder, stabilized (i.e., not worsening) state of a disorder, amelioration or palliation of the disorder, whether detectable or undetectable. “Treatment” can also mean prolonging survival as compared to expected survival in the absence of treatment.

Evaluation of Penetration Enhancers

Penetration enhancers (PE) for the formulation were evaluated to enhance the delivery of Compound A through skin by an ex vivo permeation assay using pig ear skin as a surrogate for human skin.

Compound A was formulated in pH 5 acetate buffer containing various penetration enhancers (see Table 2). Formulations were delivered to the skin over a period of 6 hours and drug permeation was assessed by UPLC-MS/MS analysis of tape-stripped skin and receptor fluid. Receptor fluid samples were collected for analysis at 3 and 6 hour time points.

Compound A was dissolved at saturation in various vehicles containing PE in pH 5 acetate buffer (see Table 2). An excess amount of Compound A and the corresponding vehicle were mixed by rotary mixer at room temperature for 24 hours and subsequent filtration through 0.22 μm cellulose acetate membrane. Concentration of Compound A was measured by UPLC.

TABLE 2
Compound A Concentration in Evaluated Formulations.

| | Compound A |
|---|---|
| | Concentration in |
| | Formulation |
| Vehicle | (mg/mL) |
| pH5 acetate buffer + 20% propylene glycol (PG) | 2.82 |
| pH5 acetate buffer + 10% urea | 3.04 |
| pH5 acetate buffer + 15% TWEEN ® 80 | 1.56 |
| pH5 acetate buffer + 10% dimethylisosorbide (DMI) | 3.18 |
| pH5 acetate buffer + 10% Transcutol ® (TC) | 2.99 |
| Water + 20% N-methyl-2-pyrollidone (NMP) | 1.13 |

A method for evaluating Granzyme B inhibitor (Compound A) permeation is described in Example 1.

Burn Wound Healing

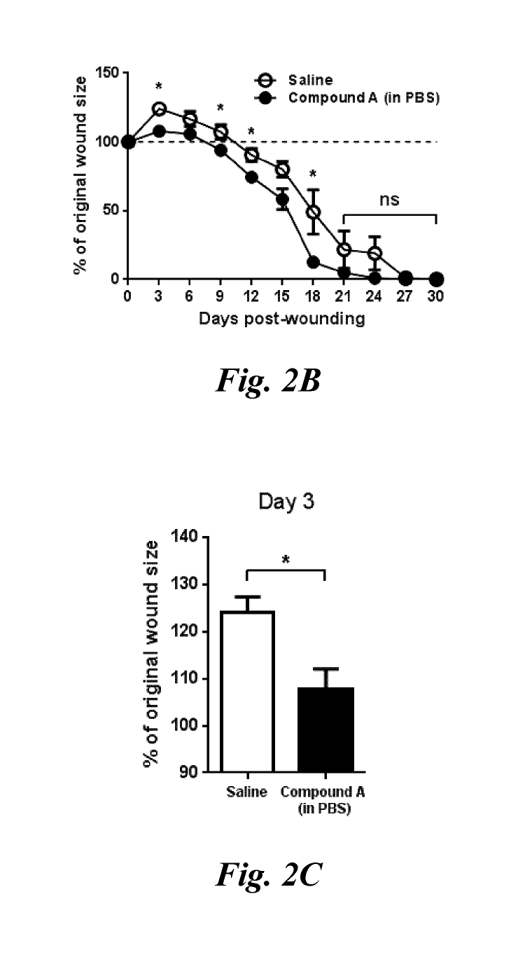

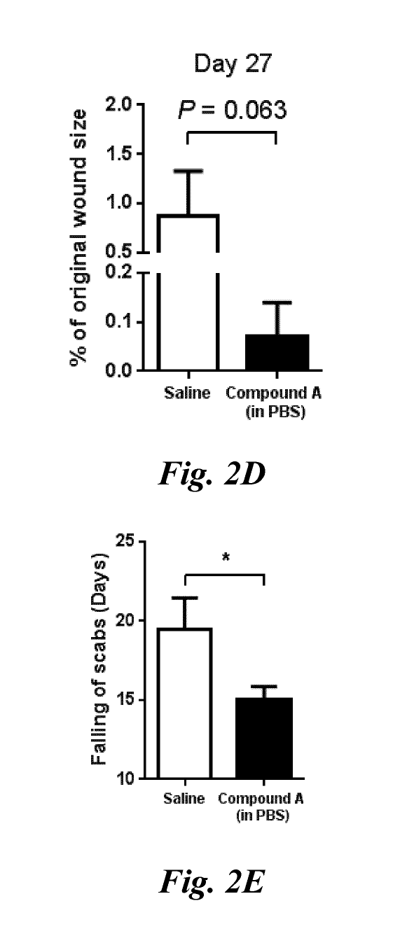

The formulations and methods of the invention are effective for the treatment of burns and for burn wound healing. The advantages of representative formulations of the invention (injectable solutions and topical gels) compared to control are illustrated in FIGS. 2-4.

FIG. 2A compares images of wounds (mice) over time (Day 0 to Day 30) after subcutaneous injection of saline (control) and a representative formulation of the invention that includes Compound A demonstrating attenuation of initial burn expansion in the zone of stasis and improved burn wound healing for the representative formulation. FIG. 2B is a graphic illustration of the comparative wound healing shown in FIG. 2A (percent of original wound size as a function of days post-wounding). FIG. 2C compares wound healing at Day 3 of the comparative wound healing shown in FIG. 2A (percent of original wound size for control (saline) and the representative formulation of the invention that includes Compound A (PBS)). FIG. 2D compares wound healing at Day 27 of the comparative wound healing shown in FIG. 2A (percent of original wound size for control (saline) and the representative formulation of the invention that includes Compound A (PBS)). FIG. 2E compares falling of scabs (days) in the comparative wound healing shown in FIG. 2A (falling of scabs (days) for control (saline) and the representative formulation of the invention that includes Compound A (PBS)).

In the studies described herein, all mice were initially anesthetized using mixture of isoflurane/oxygen (Iso about 2-2.5%/oxygen flow rate of about 1.5-2 L/min) in an induction chamber, then moved to a Baines system with a 1-2.5%/1.5-2 L/min flow rate. During anesthesia the mice were placed on a warming pad with a nose cone over the mice to maintain the anesthetic state. The toe nails were trimmed with clippers. A metal rod (25 g, 1 cm in diameter) was heated to 95-100° C. by submersion in boiling water for 3-5 min. The rod was immediately positioned vertically for 6 seconds without additional pressure on the dorsal skin of mice that had also been depilated 3 days before wounding. This procedure induces a deep partial thickness thermal injury (degree IIb). The severity of the thermal injury is classified according to Exp Dermatol. 2010; 19(9):777-783.

The results for the injectable formulation demonstrate the effectiveness of this representative formulation for treating burns and for burn wound healing.

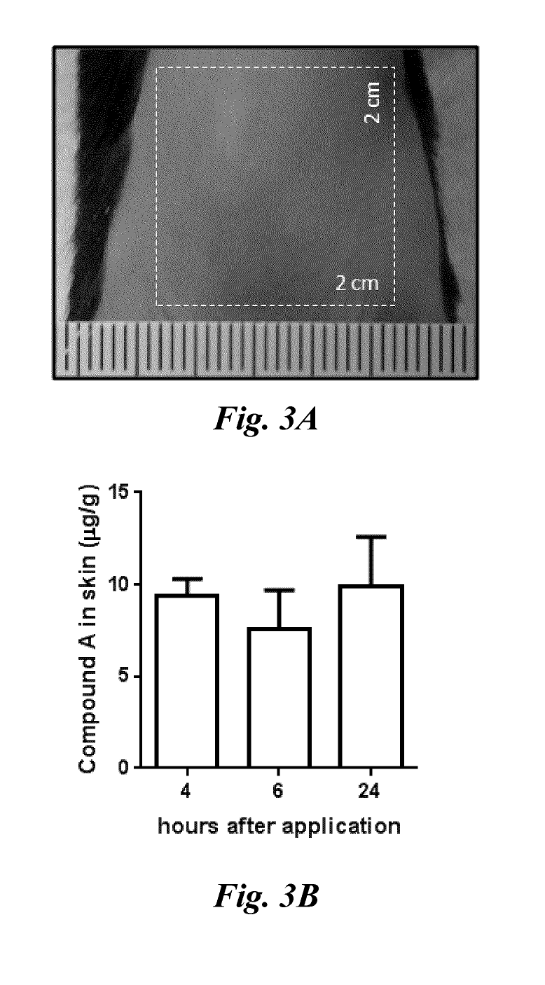

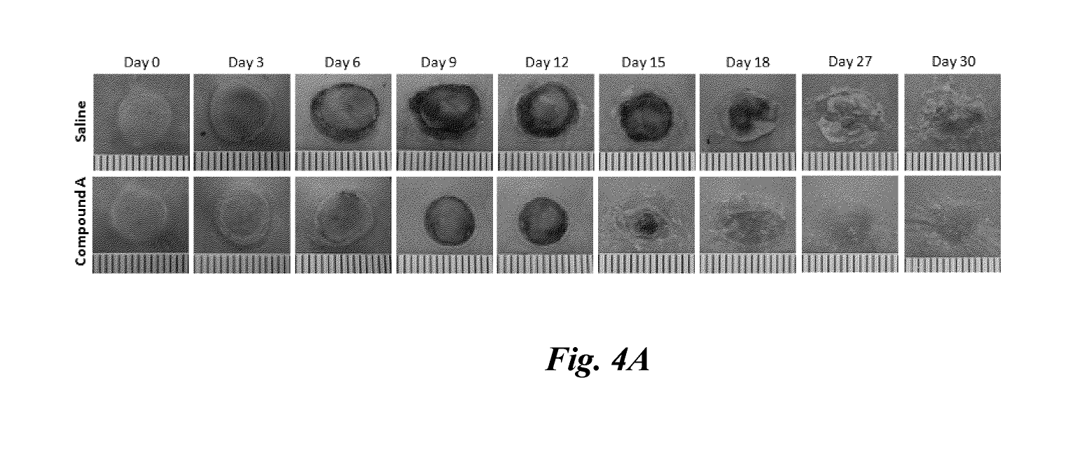

FIG. 3A is an image of skin to which a volume (50 μL) of a representative formulation of the invention that includes Compound A was applied. FIG. 3B compares concentration of Compound A (μg/μL) in the skin at 4, 6, and 24 hours after application of a volume (50 μL) of a representative formulation of the invention that includes Compound A as described above for FIG. 3A. The results demonstrate that Compound A is stable and effectively retained in the skin to which the compound was applied.

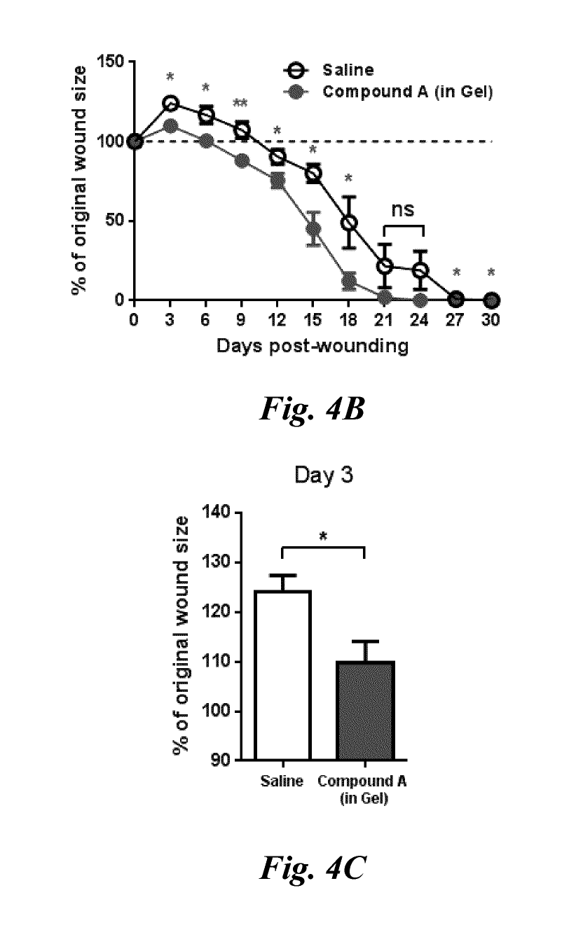

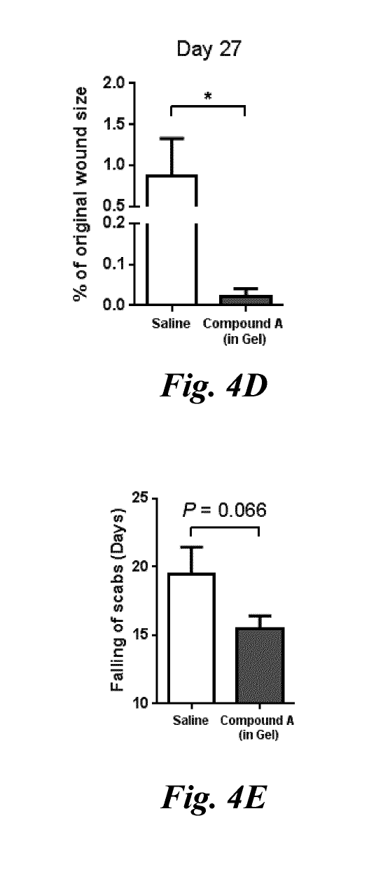

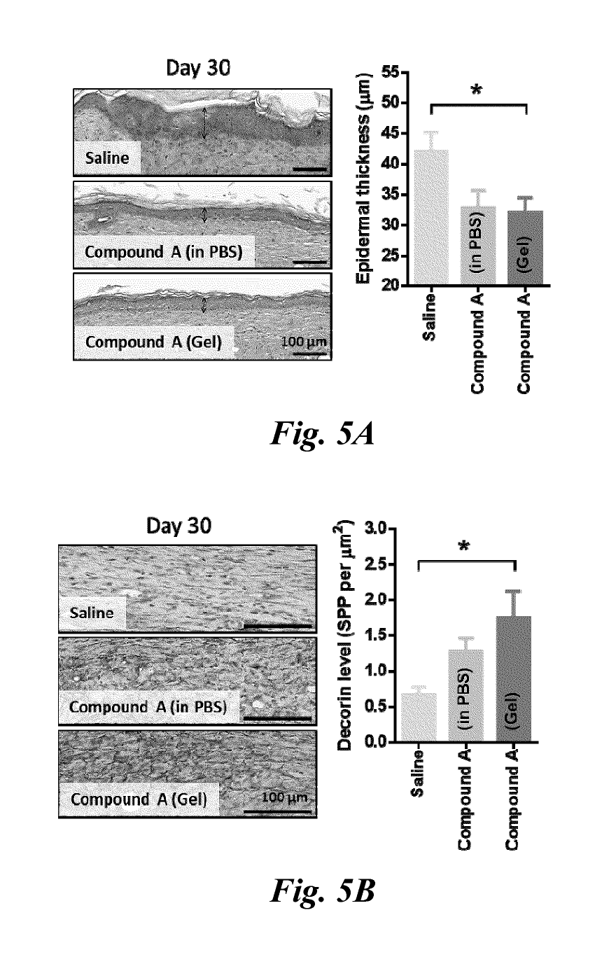

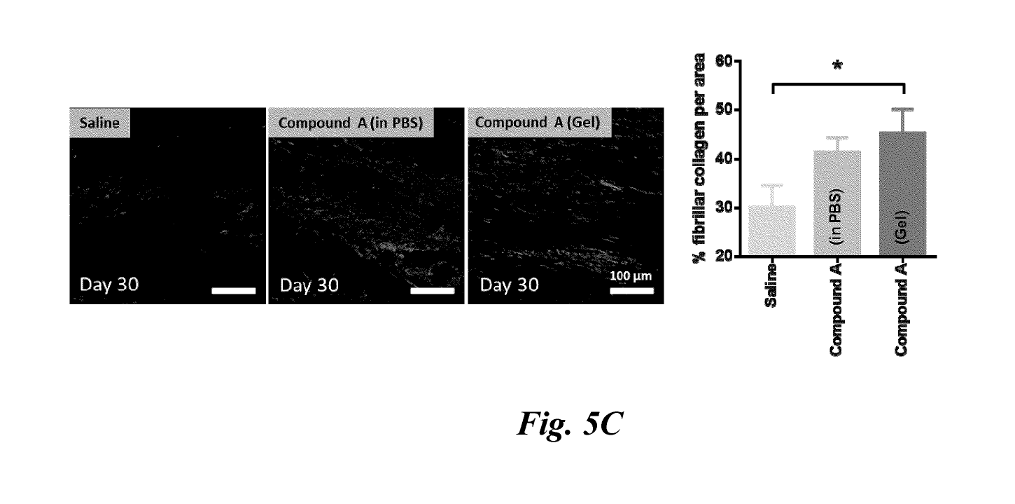

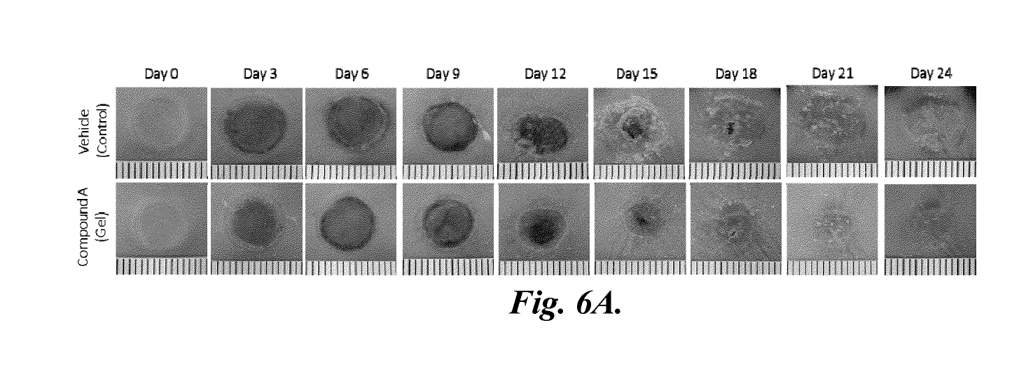

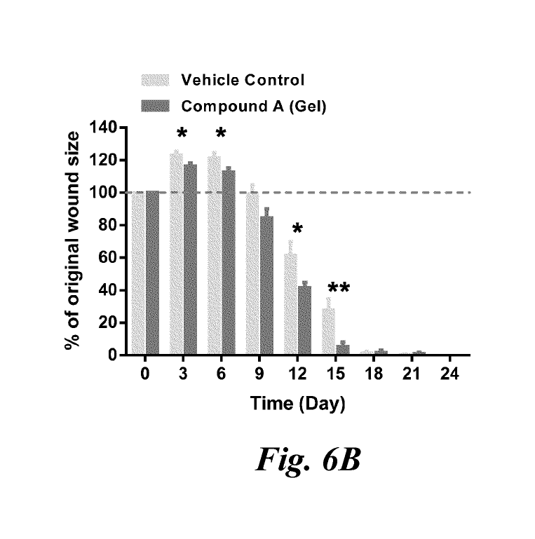

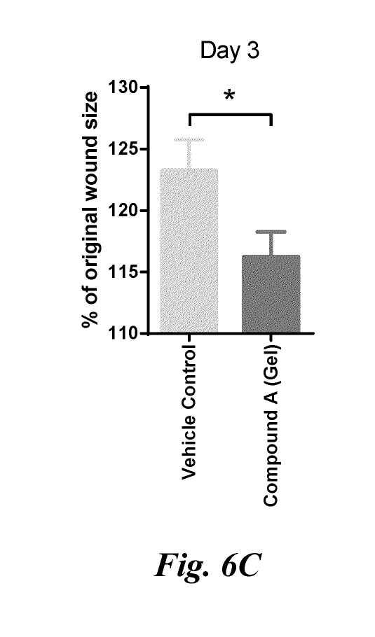

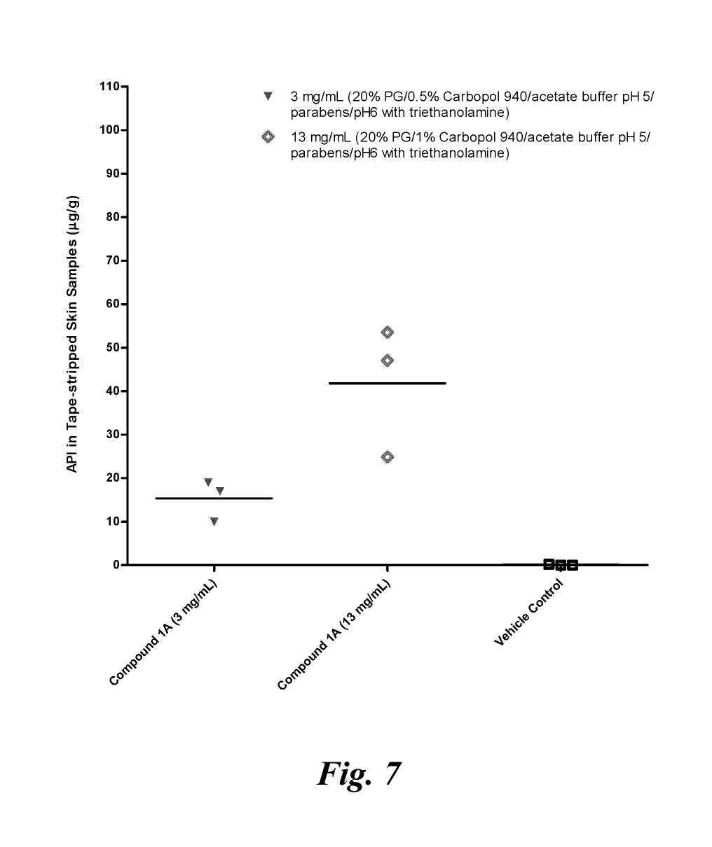

FIG. 4A compares images of wounds over time (Day 0 to Day 30) after subcutaneous injection of saline (control) and topical application of a representative formulation of the invention that includes Compound A (gel) demonstrating attenuation of initial burn expansion in the zone of stasis and improved burn wound healing for the representative formulation. FIG. 4B is a graphic illustration of the comparative wound healing shown in FIG. 4A (percent of original wound size as a function of days post-wounding). FIG. 4C compares wound healing at Day 3 of the comparative wound healing shown in FIG. 4A (percent of original wound size for control (saline) and the representative formulation of the invention that includes Compound A (gel)). FIG. 4D compares wound healing at Day 27 of the comparative wound healing shown in FIG. 4A (percent of original wound size for control (saline) and the representative formulation of the invention that includes Compound A (gel)). FIG. 4E compares falling of scabs (days) in the comparative wound healing shown in FIG. 4A (falling of scabs (days) for control (saline) and the representative formulation of the invention that includes Compound A (gel)). FIGS. 5A, 5B, and 5C demonstrate that Compound A (gel) improves quality of healed wound measured by different parameters (epidermal thickness, decorin level in the wound and collagen level in the wound). FIGURE A compares images of wounds over time (Day 0 to Day 24) after topical application of vehicle (control) and a representative formulation of the invention that includes Compound A (gel) in a repeated study demonstrating attenuation of initial burn expansion in the zone of stasis and improved burn wound healing for the representative formulation. FIG. 6B is a graphic illustration of the comparative wound healing shown in FIG. 6A (percent of original wound size as a function of days post-wounding). FIG. 6C compares wound healing at Day 3 of the comparative wound healing shown in FIG. 6A (percent of original wound size for control (vehicle) and the representative formulation of the invention that includes Compound A (gel)).

The results for the topical formulation demonstrate the effectiveness of this representative formulation for treating burns and for burn wound healing.

### Abbreviations

As used herein, the following abbreviations have the indicated meanings.

¹H NMR: proton nuclear magnetic resonance

¹⁹F NMR: fluorine-19 nuclear magnetic resonance

% Inh: Percent inhibition

Ac-IEPD-AMC: acetyl-isoleucyl-glutamyl-prolyl-aspartyl-(7-amino-4-methylcoumarin) substrate

ACN: acetonitrile

BHET: bis-2-hydroxyethyl-terephthalate

Boc: tert-butoxycarbonyl

BSA: Bovine serum albumin

CHAPS: 3-[(3-cholamidopropyl)dimethylammonio]-1-propanesulfonate

DAPI: 4′,6-diamidino-2-phenylindole

DCM: dichloromethane

DIPEA: diisopropylethylamine

DMAP: 4-dimethylaminopyridine

DMF: dimethylformamide

DMSO: dimethylsulfoxide

DMSO-d6: dimethylsulfoxide-d6

DTT: dithiothreitol

EDC: 1-(3-dimethylaminopropyl)-3-ethylcarbodiimide hydrochloride

EDTA: 2-({2-[Bis(carboxymethyl)amino]ethyl}(carboxymethyl)amino)acetic acid

ESI: Electrospray ionization

EtOAc: ethyl acetate

eq.: equivalent(s)

GzmB: Granzyme B

HATU: 2-(7-aza-1H-benzotriazole-1-yl)-1,1,1,1-tetramethyluronium hexafluorophosphate

HCl: hydrochloric acid

HEPES: 4-(2-hydroxyethyl)-1-piperazineethanesulfonic acid

hGzmB: human Granzyme B

HPLC: high performance liquid chromatography

HOBt: 1-hydroxy-benzotriazol

IC₅₀: inhibitory concentration that provides 50% inhibition

LC/MS: liquid chromatography/mass spectrometry

MeOH: methanol

mGzmB: murine Granzyme B

MS: mass spectrometry

m/z: mass to charge ratio.

Oxyma: ethyl 2-cyano-2-(hydroxyimino)acetate

PBS: phosphate buffered saline (pH 7.4)

RPM: revolution per minute

RT: room temperature

tert-BuOH: tert-butyl alcohol

THF: tetrahydrofuran

TFA: trifluoroacetic acid

wt %: weight percent

The following examples are provided for the purpose of illustrating, not limiting, the invention.

### EXAMPLES

### General Methods A-C

Representative compounds of the invention were prepared according to Methods A to C as described below and illustrated in FIGS. 9-11.

It will be appreciated that in the following general methods and preparation of synthetic intermediates, reagent levels and relative amounts or reagents/intermediates can be changed to suit particular compounds to be synthesized, up or down by up to 50% without significant change in expected results.

Method A: General Method for Deprotection Followed by Coupling Reaction Using EDC/HOBt/DIPEA.

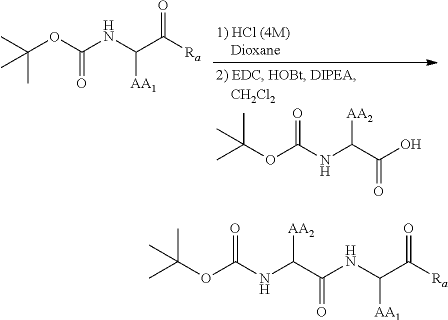

HCl Solution in dioxane (4M, 5 ml) was added to respective carbamate compound (0.125 mmol) and stirred for 2 hrs at RT. The reaction mixture was concentrated to dryness under vacuum and swapped with MeOH (5 ml) three times. Resulting residue was dried well under vacuum and subjected to next reaction as it was. The residue obtained above, respective acid moiety (0.125 mmol), EDC (0.19 mmol), HOBt (0.16 mmol) and DIPEA (0.5 mmol) were stirred in anhydrous DCM (5 ml) for 16 hrs. The reaction mixture was concentrated under vacuum to give the crude product which was purified on a C18 column using 10-50% MeOH in water to yield product as an off-white solid (35-55%).

Method B: General Method for Deprotection Followed by Reaction with Anhydride.

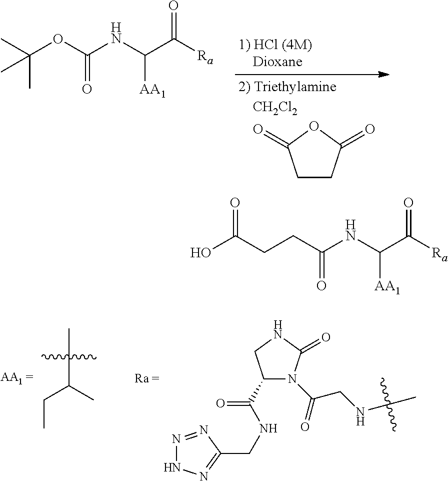

HCl Solution in dioxane (4M, 5 ml) was added to a representative Boc-protected compound (0.125 mmol) and stirred for 2 hrs at RT. The reaction mixture was concentrated to dryness under vacuum and washed with MeOH (5 ml) three times. The resulting residue was dried well under vacuum and subjected to next reaction as it was. The residue obtained above, the respective anhydride moiety (0.125 mmol), and triethylamine (0.5 mmol) were added to anhydrous DCM (5 mL) and stirred for 16 hrs. The mixture was concentrated under vacuum to give the crude product which was purified on a C18 column using 10-50% MeOH in water to yield product as an off-white solid (40-60%).

Method C: General Method for Deprotection Followed by Reaction with Anhydride.

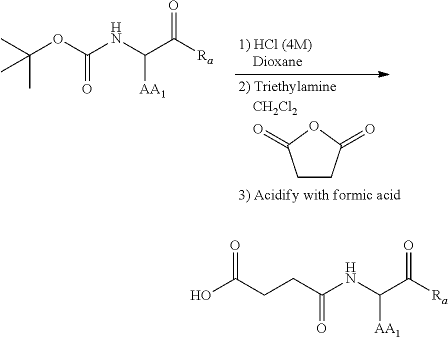

This method is an improved procedure for the method B. HCl Solution in dioxane (4M, 5 ml) was added to a representative Boc-protected compound (0.125 mmol) and stirred for 2 hrs at RT. The reaction mixture was concentrated to dryness under vacuum and swapped with MeOH (5 ml) three times. The resulting residue was dried well under vacuum and subjected to next reaction as it was. The residue obtained above, the respective anhydride moiety (0.19 mmol, 1.5 eq.), and triethylamine (0.5 mmol, 4 eq.) were added to anhydrous DCM (5 mL) and stirred for 16 hrs. The mixture was acidified with formic acid and then concentrated under vacuum to give the crude product which was purified on a C18 column using 25-65% MeOH in water to yield product as an off-white solid (30-80%).

### Synthetic Intermediates

The following is a description of synthetic intermediates (I-1 to I-4) useful for making representative compounds of the invention.

### Intermediate I-1

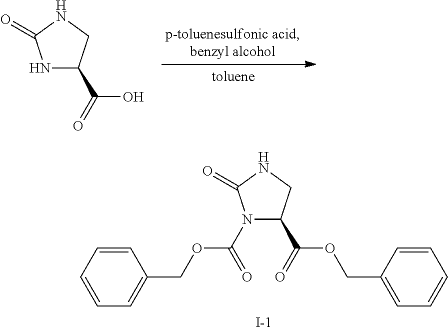 [SMILES: O=C1NC[C@@H](C(=O)O)N1 ; O=C(OCc1ccccc1)[C@@H]1CNC(=O)N1C(=O)OCc1ccccc1 ; [I-]]

### (S)-Dibenzyl 2-oxoimidazolidine-1,5-dicarboxylate (I-1)

(S)-(−)-1-Z-2-Oxo-5-imidazolidinecarboxylic acid (2.5 g, 9.461 mmol, 1 eq.), para-toluenesulfonic acid (360 mg, 1.892 mmol, 0.2 eq.) and benzyl alcohol (2.39 mL, 23.12 mmol, 2.4 eq.) were dissolved in toluene (25 mL) in a round bottom flask equipped with a Dean-stark apparatus and a condenser. The reaction was heated to reflux for 24 hrs and then allowed to come to RT. It was then washed with a saturated solution of NaHCO₃ solution (1×25 mL) and the aqueous layer was re-extracted with ethyl acetate (1×25 mL). The combined organic layers were dried over Na₂SO₄ and concentrated. The product was then purified by column chromatography using 15% to 70% ethyl acetate in hexanes as the eluent to give (S)-dibenzyl 2-oxoimidazolidine-1,5-dicarboxylate (I-1) as a white solid (1.50 g, 45%). ¹H NMR (400 MHz, CDCl₃) δ 3.41 (1H, dd, J=4, 7 Hz), 3.74 (1H, t, J=10 Hz), 4.80 (1H, dd, J=4, 10 Hz), 5.10-5.17 (2H, m), 5.18-5.25 (2H, m), 6.08 (1H, s), 7.27-7.39 (10H, m), MS (LC/MS) m/z observed 354.82, expected 355.13 [M+H].

### Intermediate I-2

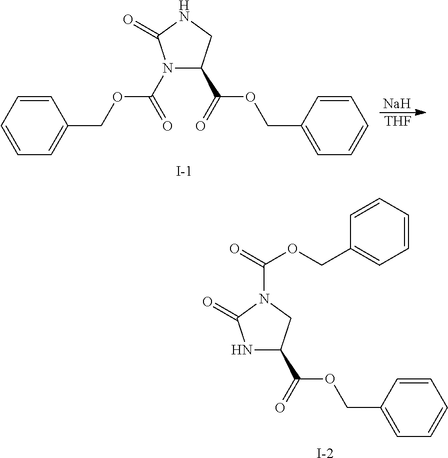

### (S)-2-Oxo-imidazolidine-1,4-dicarboxylic acid dibenzyl ester (I-2)

A slurry of 60% NaH in oil (24.8 mg, 0.621 mmol, 1.1 eq.) was added to a solution of I-1 (200 mg, 0.564 mmol, 1 eq.) in anhydrous THF (25 mL) at 0° C. under N2. The reaction was left at 0° C. for 5 min and then allowed to warm to 10° C. and was stirred for an additional hour at 10° C. The reaction was added AcOH (0.5 mL) and the solvent was evaporated. The product was then purified by column chromatography using 15% to 70% ethyl acetate in hexanes as the eluent to give (S)-2-oxo-imidazolidine-1,4-dicarboxylic acid dibenzyl ester (I-2) as a white solid (110.5 mg, 55%). ¹H NMR (400 MHz, CDCl₃) δ 4.06-4.17 (2H, m), 4.27 (1H, dd, J=5, 9 Hz), 5.21 (2H, s), 5.27 (2H, s), 5.67 (1H, s), 7.32-7.45 (10H, m), MS (LC/MS) m/z observed 354.86, expected 355.13 [M+H].

### Intermediate I-3

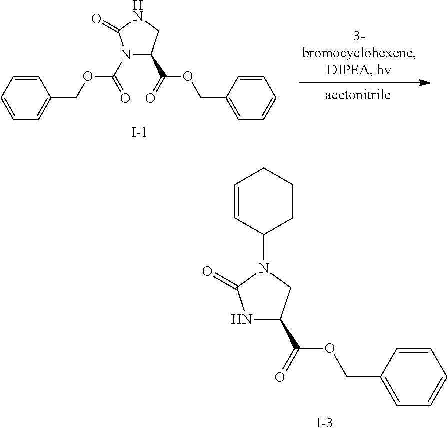

### (4S)-Benzyl 1-(cyclohex-2-en-1-yl)-2-oxoimidazolidine-4-carboxylate (I-3)

I-1 (300 mg, 0.846 mmol, 1 eq.) was dissolved in acetonitrile (7.5 mL) in a microwave vial. DIPEA (2.9 mL, 16.93 mmol, 20 eq.) and 3-bromocyclohexene (1.9 mL, 16.93 mmol, 20 eq.) were then added to the vial and it was microwaved at 100° C. for 2 hrs. The product was then purified by column chromatography using 5% to 70% ethyl acetate in hexanes as the eluent to give (4S)-benzyl 1-(cyclohex-2-en-1-yl)-2-oxoimidazolidine-4-carboxylate (I-3) as a white solid (220 mg, 87%). MS (LC/MS) m/z observed 300.80, expected 301.16 [M+H]. Compound was confirmed using LC/MS and moved to next step as it was.

### Intermediate I-4

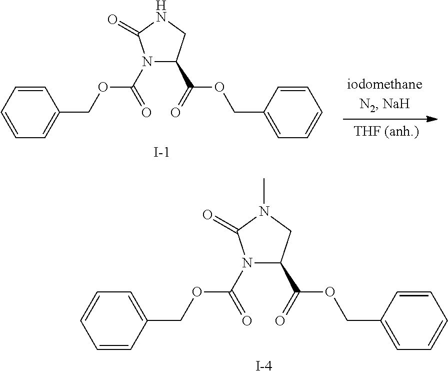

### (S)-Dibenzyl 3-methyl-2-oxoimidazolidine-1,5-dicarboxylate (I-4)

To a stirring solution of I-1 (450 mg, 1.269 mmol, 1 eq.) in anhydrous THF (20 mL) was added iodomethane (0.8 mL, 12.69 mmol, 10 eq.) under N₂. The reaction mixture was cooled to 0° C. and a slurry of 60% NaH in oil (60.1 mg, 1.524 mmol, 1.1 eq.) was added. The reaction was kept at 0° C. for 30 minutes and was then added a saturated solution of ammonium chloride (1 mL). It was then diluted with ethyl acetate (30 mL) and washed a 20% sodium thiosulphate solution (1×25 mL). The combined organic layers were dried over Na₂SO₄ and concentrated. The product was then purified by column chromatography using 10% to 70% ethyl acetate in hexanes as the eluent to give (S)-dibenzyl 3-methyl-2-oxoimidazolidine-1,5-dicarboxylate (I-4) as a colorless glass (180 mg, 38%). MS (LC/MS) m/z observed 368.81, expected 369.15 [M+H]. Compound was confirmed using LC/MS and moved to next step as it was.

### Representative Granzyme B Inhibitor Compounds

The following is a description of the preparation of representative Granzyme B inhibitor compounds of the invention.

Examples C1-C6 were prepared by the representative synthetic pathway illustrated schematically in FIG. 3.

### Example C1

### 4-(((2S,3S)-1-((2-((S)-5 #(2H-Tetrazol-5-yl)methyl)carbamoyl)-2-oxoimidazolidin-1-yl)-2-oxoethyl)amino)-3-methyl-1-oxopentan-2-yl)amino)-4-oxobutanoic acid

A solution of 1-2 (100 mg, 0.282 mmol, 1 eq.) in anhydrous THF (2 mL) was cooled to −50° C. under N₂. Potassium tert-butoxide (31.6 mg, 0.282 mmol, 1 eq.) was then added, followed by Boc-glycine N-hydroxysuccinimide ester (76.7 mg, 0.282 mmol, 1 eq.) and the reaction mixture was slowly allowed to warm up to −10° C. and it was stirred at that temperature for 1 h. Analysis of the reaction mixture by TLC showed the presence of starting material left. The reaction mixture was cooled to −50° C. and potassium tert-butoxide (31.6 mg, 0.282 mmol, 1 eq.), followed by Boc-glycine N-hydroxysuccinimide ester (76.7 mg, 0.282 mmol, 1 eq.) were added and the reaction mixture was slowly allowed to warm up to −10° C. and it was stirred at that temperature for 1 h. TLC showed disappearance of the starting material. The reaction mixture was added AcOH (0.5 mL) and the solvent was evaporated. The residue was submitted to a column chromatography using 15% to 50% ethyl acetate in hexanes as the eluent to give the mixture of related compounds (S)-dibenzyl 3-(2-((tert-butoxycarbonyl)amino)acetyl)-2-oxoimidazolidine-1,4-dicarboxylate and (S)-1-((benzyloxy)carbonyl)-3-(2-((tert-butoxycarbonyl)amino)acetyl)-2-oxoimidazolidine-4-carboxylic acid (113 mg). (LC/MS) m/z observed 534.60, expected 534.19 [M+H] for the benzyl ester and (LC/MS) m/z observed 422.03, expected 422.16 [M+H] for the acid. Compounds were confirmed using LC/MS and moved to next step as they were.

The mixture of related compounds (S)-dibenzyl 3-(2-((tert-butoxycarbonyl)amino)acetyl)-2-oxoimidazolidine-1,4-dicarboxylate and (S)-1-((benzyloxy)carbonyl)-3-(2-((tert-butoxycarbonyl)amino)acetyl)-2-oxoimidazolidine-4-carboxylic acid (255 mg) was dissolved in methanol (10 mL) and palladium on charcoal 10% by wt (10 mg) was added to the solution under N₂. The flask was then flushed with H₂ and H₂ was bubbled into the reaction mixture for 4 hrs. The flask was flushed with N₂ and the reaction mixture was filtered over celite. The solids were washed with methanol (3×15 mL) and the filtrate and washings were then concentrated to give (S)-3-(2-((tert-butoxycarbonyl)amino)acetyl)-2-oxoimidazolidine-4-carboxylic acid as a brown oil (143.2 mg, quantitative). (LC/MS) m/z observed 287.80, expected 288.12 [M+H]. Compound was confirmed using LC/MS and moved to next step as it was.

(S)-tert-Butyl (2-(5-(((2H-tetrazol-5-yl)methyl)carbamoyl)-2-oxoimidazolidin-1-yl)-2-oxoethyl)carbamate was prepared from (S)-3-(2-((tert-butoxycarbonyl)amino)acetyl)-2-oxoimidazolidine-4-carboxylic acid and (2H-tetrazol-5-yl)methyl-amine using method A in DMF but without HCl treatment. MS (LC/MS) m/z observed 368.90, expected 369.16 [M+H]. Compound was confirmed using LC/MS and moved to next step as it was.

tert-Butyl ((2S,3S)-1-((2-((S)-5-(((2H-tetrazol-5-yl)methyl)carbamoyl)-2-oxoimidazolidin-1-yl)-2-oxoethyl)amino)-3-methyl-1-oxopentan-2-yl)carbamate was prepared from (S)-tert-butyl (2-(5-(((2H-tetrazol-5-yl)methyl)carbamoyl)-2-oxoimidazolidin-1-yl)-2-oxoethyl)carbamate and Boc-L-isoleucine using method A in DMF. MS (LC/MS) m/z observed 481.80, expected 482.25 [M+H]. Compound was confirmed using LC/MS and moved to next step as it was.

Title compound 4-(((2S,3S)-1-((2-((S)-5-(((2H-tetrazol-5-yl)methyl)carbamoyl)-2-oxoimidazolidin-1-yl)-2-oxoethyl)amino)-3-methyl-1-oxopentan-2-yl)amino)-4-oxobutanoic acid (C1) was prepared from tert-butyl ((2S,3S)-1-((2-((S)-5-(((2H-tetrazol-5-yl)methyl)carbamoyl)-2-oxoimidazolidin-1-yl)-2-oxoethyl)amino)-3-methyl-1-oxopentan-2-yl)carbamate and succinic anhydride using method I. ¹H NMR (400 MHz, DMSO-d6) δ 0.80 (3H, t, J=7 Hz), 0.84 (3H, d, J=7 Hz), 1.08 (1H, m), 1.42 (1H, m), 1.70 (1H, m), 2.32-2.45 (4H, m), 3.22 (1H, dd, J=3, 9 Hz), 3.65 (1H, t, J=10 Hz), 4.16-4.26 (2H, m), 4.48 (1H, t, J=6 Hz), 4.53 (1H, m), 4.63 (1H, dd, J=6, 16 Hz), 4.67 (1H, dd, J=4, 10 Hz), 7.80 (1H, s), 7.90 (1H, d, J=9 Hz), 8.12 (1H, m), 8.94 (1H, t, J=6 Hz), MS (LC/MS) m/z observed 481.74, expected 482.21 [M+H]

### Example C2

### 4-(((2S,3S)-1-((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)amino)-4-oxobutanoic acid

A solution of 1-3 (220 mg, 0.733 mmol, 1 eq.) (prepared as described below) in anhydrous tetrahydrofuran (THF) (15 mL) was cooled to −50° C. under nitrogen. Potassium tert-butoxide (98 mg, 0.880 mmol, 1.2 eq.) was then added, followed by Boc-glycine N-hydroxysuccinimide ester (240 mg, 0.880 mmol, 1.2 eq.) and the reaction mixture was slowly allowed to warm up to −10° C. and it was stirred at that temperature for 1 h. Analysis of the reaction mixture by thin layer chromatography (TLC) showed the presence of starting material left. The reaction mixture was cooled to −50° C. and potassium tert-butoxide (25 mg, 0.219 mmol, 0.25 eq.), followed by Boc-glycine N-hydroxysuccinimide ester (60 mg, 0.219 mmol, 0.25 eq.) were added and the reaction mixture was slowly allowed to warm up to −10° C. and it was stirred at that temperature for 1 h. TLC showed disappearance of the starting material. The reaction mixture was added acetic acid (AcOH) (1 mL) and the solvent was evaporated. The residue was submitted to a column chromatography using 10% to 50% ethyl acetate in hexanes as the eluent to give (4S)-benzyl 3-(2-((tert-butoxycarbonyl)amino)acetyl)-1-(cyclohex-2-en-1-yl)-2-oxoimidazolidine-4-carboxylate (150 mg). MS (LC/MS) m/z observed 458.02, expected 458.53 [M+H]. The compound was confirmed using LC/MS and moved to next step as it was.

(4S)-Benzyl 3-(2-((tert-butoxycarbonyl)amino)acetyl)-1-(cyclohex-2-en-1-yl)-2-oxoimidazolidine-4-carboxylate (150 mg) was dissolved in methanol (10 mL) and palladium on charcoal 10% by wt (10 mg) was added to the solution under nitrogen. The flask was then flushed with hydrogen and hydrogen was bubbled into the reaction mixture for 16 hrs. The flask was flushed with nitrogen and the reaction mixture was filtered over CELITE®. The solids were washed with methanol (3×15 mL) and the filtrate and washings were then concentrated to give (S)-3-(2-((tert-butoxycarbonyl)amino)acetyl)-1-cyclohexyl-2-oxoimidazolidine-4-carboxylic acid as a yellow oil (120 mg, quantitative). (LC/MS) m/z observed 369.96, expected 370.20 [M+H]. The compound was confirmed using LC/MS and moved to next step as it was.

(S)-tert-Butyl (2-(5-(((2H-tetrazol-5-yl)methyl)carbamoyl)-3-cyclohexyl-2-oxoimidazolidin-1-yl)-2-oxoethyl)carbamate was prepared from (S)-3-(2-((tert-butoxycarbonyl)amino)acetyl)-1-cyclohexyl-2-oxoimidazolidine-4-carboxylic acid and (2H-tetrazol-5-yl)methyl-amine in dimethylformamide (DMF) but without hydrochloric acid (HCl) treatment. MS (LC/MS) m/z observed 450.97, expected 451.24 [M+H]. The compound was confirmed using LC/MS and moved to next step as it was.

tert-Butyl ((2S,3S)-1-((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)carbamate was prepared from (S)-tert-butyl (2-(5-(((2H-tetrazol-5-yl)methyl)carbamoyl)-3-cyclohexyl-2-oxoimidazolidin-1-yl)-2-oxoethyl)carbamate and Boc-L-isoleucine using method A in DMF. MS (LC/MS) m/z observed 564.51, expected 564.66 [M+H]. The compound was confirmed using LC/MS and moved to next step as it was.

Title Compound 4-(((2S,3S)-1-((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)amino)-4-oxobutanoic acid was prepared from tert-butyl ((2S,3S)-1-((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)carbamate by treatment with succinic anhydride. HCl solution in dioxane (4M, 5 ml) was added to the Boc-protected compound (0.125 mmol) and stirred for 2 hrs at RT. The reaction mixture was concentrated to dryness under vacuum and swapped with methanol (MeOH) (5 ml) three times. The resulting residue was dried well under vacuum and subjected to next reaction as it was. The residue obtained above, the respective anhydride moiety (0.19 mmol, 1.5 eq.), and triethylamine (0.5 mmol, 4 eq.) were added to anhydrous dichloromethane (DCM) (5 mL) and stirred for 16 hrs. The mixture was acidified with formic acid and then concentrated under vacuum to give the crude product which was purified on a C18 column using 25-65% MeOH in water to yield the title compound. ¹H NMR (400 MHz, DMSO-d₆) δ 0.80 (3H, t, J=7 Hz), 0.84 (3H, d, J=7 Hz), 1.03-1.12 (2H, m), 1.21-1.45 (5H, m), 1.50-1.80 (6H, m), 2.32-2.45 (4H, m), 3.26 (1H, dd, J=3, 9 Hz), 3.58 (1H, m), 3.67 (1H, t, J=10 Hz), 4.16-4.26 (2H, m), 4.48-4.65 (4H, m), 7.90 (1H, d, J=9 Hz), 8.15 (1H, t, J=6 Hz), 8.98 (1H, t, J=6 Hz), MS (LC/MS) m/z observed 563.95, expected 564.29 [M+H].

Compound C2 has the following structure:

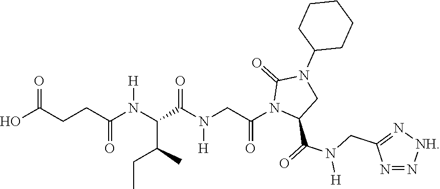 [SMILES: CC[C@H](C)[C@H](NC(=O)CCC(=O)O)C(=O)NCC(=O)N1C(=O)N(C2CCCCC2)C[C@H]1C(=O)NCc1nn[nH]n1]

Compound C2 prepared as described above is referred to herein as Compound A.

In an alternative preparation, Compound C2 was isolated by crystallization as follows (the volumes noted below are related to the weight of compound produced and are varied depending on the amount of product). The white solid obtained after evaporation was suspended in a mixture of boiling H₂O (92 mL) and 1 M HCl (23 mL). Hot isopropanol (iPrOH) was added in small portions until the white solid was completely dissolved (60 mL). The solution was slowly cooled to room temperature and then 4° C. whereupon a white precipitate formed. This solid was isolated by filtration and washed with cold 1:1 H₂O/iPrOH, giving 6.32 g of white crystalline solid. On standing, a second crop of crystals were isolated from the mother liquor (0.29 g). The combined crops of crystals resulted in 6.61 g (50% yield, 3 steps) of 4-(((2S,3S)-1-((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)amino)-4-oxobutanoic acid as a 3:2 solvate with iPrOH. Compound C2 prepared as described above is referred to herein as Compound A1.

### Example C3

### 4-(((2S,3S)-1-((2-((S)-5-(((2H-tetrazol-5-yl)methyl)carbamoyl)-3-methyl-2-oxoimidazolidin-1-yl)-2-oxoethyl)amino)-3-methyl-1-oxopentan-2-yl)amino)-4-oxobutanoic acid

I-4 (350 mg, 0.951 mmol, 1 eq.) was treated with 30% HBr in acetic acid (1.4 mL) for 20 minutes at RT. The solvent was then concentrated to dryness and the residue was submitted to a reverse phase column chromatography using 0 to 50% MeOH in water as the eluent. The obtained product was dissolved in anhydrous THF (15 mL) was cooled to −50° C. under N₂. Potassium tert-butoxide (160 mg, 1.427 mmol, 1.5 eq.) was then added, followed by Boc-glycine N-hydroxysuccinimide ester (390 mg, 1.427 mmol, 1.5 eq.) and the reaction mixture was slowly allowed to warm up to −10° C. and it was stirred at that temperature for 1 h. Analysis of the reaction mixture by TLC showed disappearance of the starting material. The reaction mixture was added AcOH (1.5 mL) and the solvent was evaporated. The residue was submitted to a column chromatography using 10% to 50% ethyl acetate in hexanes as the eluent to give (S)-benzyl 3-(2-((tert-butoxycarbonyl)amino)acetyl)-1-methyl-2-oxoimidazolidine-4-carboxylate (100 mg). (LC/MS) m/z observed 391.95, expected 392.18 [M+H]. Compound was confirmed using LC/MS and moved to next step as it was.

(S)-Benzyl 3-(2-((2S,3S)-2-((tert-butoxycarbonyl)amino)-3-methylpentanamido)acetyl)-1-methyl-2-oxoimidazolidine-4-carboxylate was prepared from (S)-benzyl 3-(2-((tert-butoxycarbonyl)amino)acetyl)-1-methyl-2-oxoimidazolidine-4-carboxylate and Boc-L-isoleucine using method A in DMF. MS (LC/MS) m/z observed 505.23, expected 505.27 [M+H]. Compound was confirmed using LC/MS and moved to next step as it was.

(S)-Benzyl 3-(2-((2S,3S)-2-((tert-butoxycarbonyl)amino)-3-methylpentanamido)acetyl)-1-methyl-2-oxoimidazolidine-4-carboxylate (30 mg) was dissolved in ethanol (6 mL) and palladium on charcoal 10% by wt (10 mg) was added to the solution under N₂. The flask was then flushed with H₂ and H₂ was bubbled into the reaction mixture for 4 hrs. The flask was flushed with N₂ and the reaction mixture was filtered over celite. The solids were washed with methanol (3×15 mL) and the filtrate and washings were then concentrated to give (S)-3-(2-((2S,3S)-2-((tert-butoxycarbonyl)amino)-3-methylpentanamido)acetyl)-1-methyl-2-oxoimidazolidine-4-carboxylic acid as a yellow oil (24 mg, quantitative). MS (LC/MS) m/z observed 414.98, expected 415.22 [M+H]. Compound was confirmed using LC/MS and moved to next step as it was.

tert-Butyl ((2S,3S)-1-((2-((S)-5-(((2H-tetrazol-5-yl)methyl)carbamoyl)-3-methyl-2-oxoimidazolidin-1-yl)-2-oxoethyl)amino)-3-methyl-1-oxopentan-2-yl)carbamate was prepared from (S)-3-(2-((2S,3S)-2-((tert-butoxycarbonyl)amino)-3-methylpentanamido)acetyl)-1-methyl-2-oxoimidazolidine-4-carboxylic acid and (2H-tetrazol-5-yl)methyl-amine using method A in DMF but without HCl treatment. MS (LC/MS) m/z observed 495.89, expected 496.26 [M+H]. Compound was confirmed using LC/MS and moved to next step as it was.

Title compound 4-(((2S,3 S)-1-((2-((S)-5-(((2H-tetrazol-5-yl)methyl)carbamoyl)-3-methyl-2-oxoimidazolidin-1-yl)-2-oxoethyl)amino)-3-methyl-1-oxopentan-2-yl)amino)-4-oxobutanoic acid (C3) was prepared from tert-butyl ((2S,3S)-1-((2-((S)-5-(((2H-tetrazol-5-yl)methyl)carbamoyl)-3-methyl-2-oxoimidazolidin-1-yl)-2-oxoethyl)amino)-3-methyl-1-oxopentan-2-yl)carbamate and succinic anhydride using method I. ¹H NMR (400 MHz, DMSO-d6) δ 0.71-0.85 (6H, m), 1.08 (1H, m), 1.42 (1H, m), 1.70 (1H, m), 2.32-2.41 (3H, m), 2.45-2.50 (4H, m), 3.27 (1H, m), 3.65-3.75 (2H, m), 4.08-4.26 (2H, m), 4.45-4.65 (3H, m), 7.95 (1H, m), 8.15 (1H, m), 8.95 (1H, m), MS (LC/MS) m/z observed 495.90, expected 496.23 [M+H]

### Example C4

### (S)-5-((S)-5-(((2H-Tetrazol-5-yl)methyl)carbamoyl)-3-cyclohexyl-2-oxoimidazolidin-1-yl)-4-((2S,3S)-2-(3-carboxypropanamido)-3-methylpentanamido)-5-oxopentanoic acid

(S)-(−)-1-Z-2-Oxo-5-imidazolidinecarboxylic acid (2.0 g, 7.569 mmol, 1 eq.), DMAP (92.5 mg, 0.757 mmol, 0.1 eq.) and tert-butanol (2.17 mL, 22.71 mmol, 3 eq.) were dissolved in CH₂Cl₂ (38 mL). The reaction was cooled to 0° C. and EDC (1.74 g, 9.083 mmol, 1.2 eq.) was added. The reaction was left at 0° C. for 1 h and stirred at RT for 16 hrs. The solvent was evaporated and the product was purified by normal phase column chromatography using 15% to 70% ethyl acetate in hexanes as the eluent to give (S)-1-benzyl 5-tert-butyl 2-oxoimidazolidine-1,5-dicarboxylic acid as a white solid (1.05 g, 43%). ¹H NMR (400 MHz, CDCl₃) δ 1.38 (9H, s), 3.38 (1H, dd, J=4, 7 Hz), 3.73 (1H, t, J=10 Hz), 4.63 (1H, dd, J=4, 10 Hz), 5.20-5.33 (4H, m), 6.25 (1H, s), 7.30-7.40 (5H, m), (LC/MS) m/z observed 320.82, expected 321.15 [M+H].

(S)-1-Benzyl 5-tert-butyl 2-oxoimidazolidine-1,5-dicarboxylic acid (1.05 g, 3.290 mmol, 1 eq.) was dissolved in acetonitrile (15 mL) in a microwave vial. 3-bromocyclohexene (1.89 mL, 16.45 mmol, 5 eq.) and potassium tert-butoxide (406 mg, 3.619 mmol, 1.1 eq) were then added to the vial and it was microwaved at 90° C. for 1 minute. The reaction was then quenched with AcOH (5 mL) and the solvents were concentrated. The product was then purified by reverse phase column chromatography using 5% to 80% methanol in water as the eluent to give (5S)-1-benzyl 5-tert-butyl 3-(cyclohex-2-en-1-yl)-2-oxoimidazolidine-1,5-dicarboxylate as an orange glass (496 mg, 38%). MS (LC/MS) m/z observed 400.97, expected 401.21 [M+H]. Compound was confirmed using LC/MS and moved to next step as it was.

(5S)-1-Benzyl 5-tert-butyl 3-(cyclohex-2-en-1-yl)-2-oxoimidazolidine-1,5-dicarboxylate (496 mg, 2.497 mmol) was dissolved in methanol (50 mL) and palladium on charcoal 10% by wt (50 mg) was added to the solution under N₂. The flask was then flushed with H₂ and H₂ was bubbled into the reaction mixture for 4 hrs. The flask was flushed with N₂ and the reaction mixture was filtered over celite. The solids were washed with methanol (3×50 mL) and the filtrate and washings were then concentrated to give (S)-tert-butyl 1-cyclohexyl-2-oxoimidazolidine-4-carboxylate as a yellow glass (314.3 mg, quantitative). (LC/MS) m/z observed 268.95, expected 269.19 [M+H]. Compound was confirmed using LC/MS and moved to next step as it was.

A solution of (S)-tert-butyl 1-cyclohexyl-2-oxoimidazolidine-4-carboxylate (199 mg, 0.742 mmol, 1 eq.) in anhydrous THF (5 mL) was cooled to −50° C. under N₂. Potassium tert-butoxide (83.2 mg, 0.742 mmol, 1 eq.) was then added, followed by Boc-L-glutamic acid benzyl ester N-hydroxysuccinimide ester (322.4 mg, 0.742 mmol, 1 eq.) and the reaction mixture was slowly allowed to warm up to −10° C. and it was stirred at that temperature for 1 h. Analysis of the reaction mixture by TLC showed completion of the reaction. The reaction mixture was added AcOH (1 mL) and the solvent was evaporated. The residue was submitted to a reverse phase column chromatography using 10% to 85% methanol in water as the eluent to give (S)-tert-butyl 3-((S)-5-(benzyloxy)-2-((tert-butoxycarbonyl)amino)-5-oxopentanoyl)-1-cyclohexyl-2-oxoimidazolidine-4-carboxylate as a colorless glass (230.1 mg, 53%). (LC/MS) m/z observed 587.84, expected 588.33 [M+H] Compound was confirmed using LC/MS and moved to next step as it was.

(S)-3-((S)-5-(benzyloxy)-2-((2S,3S)-2-((tert-butoxycarbonyl)amino)-3-methylpentanamido)-5-oxopentanoyl)-1-cyclohexyl-2-oxoimidazolidine-4-carboxylic acid was prepared from (S)-tert-butyl 3-((S)-5-(benzyloxy)-2-((tert-butoxycarbonyl)amino)-5-oxopentanoyl)-1-cyclohexyl-2-oxoimidazolidine-4-carboxylate and Boc-L-isoleucine using method A in DMF. MS (LC/MS) m/z observed 644.86, expected 645.35 [M+H]. Compound was confirmed using LC/MS and moved to next step as it was.

(S)-Benzyl 5-((S)-5-(((2H-tetrazol-5-yl)methyl)carbamoyl)-3-cyclohexyl-2-oxoimidazolidin-1-yl)-4-((2S,3S)-2-((tert-butoxycarbonyl)amino)-3-methylpentanamido)-5-oxopentanoate was prepared from (S)-3-((S)-5-(benzyloxy)-2-((2S,3S)-2-((tert-butoxycarbonyl)amino)-3-methylpentanamido)-5-oxopentanoyl)-1-cyclohexyl-2-oxoimidazolidine-4-carboxylic acid and (2H-tetrazol-5-yl)methyl-amine using method A in DMF but without HCl treatment. The reaction was stirred at RT for 16 hrs and then heated to 50° C. for 4 additional hours. MS (LC/MS) m/z observed 725.88, expected 726.39 [M+H]. Compound was confirmed using LC/MS and moved to next step as it was.

(S)-Benzyl 5-((S)-5-(((2H-tetrazol-5-yl)methyl)carbamoyl)-3-cyclohexyl-2-oxoimidazolidin-1-yl)-4-((2S,3S)-2-((tert-butoxycarbonyl)amino)-3-methylpentanamido)-5-oxopentanoate (39.4 mg, 0.0595 mmol) and succinic anhydride (8.9 mg, 0.0893 mmol, 1.5 eq.) were suspended in CH₂Cl₂ (5 mL). NEt₃ (0.033 mL, 0.238 mmol, 4 eq.) was added and the reaction mixture was stirred at RT for 1 hour, reacting to completion. The solvent was evaporated and the residue was dissolved in methanol (10 mL) containing AcOH (1 mL) and palladium on charcoal 10% by wt (10 mg) was added to the solution under N₂. The flask was then flushed with H₂ and H₂ was bubbled into the reaction mixture for 4 hrs. The flask was flushed with N₂ and the reaction mixture was filtered over CELITE™. The solids were washed with methanol (3×15 mL) and the filtrate and washings were then concentrated to give a residue that was submitted to preparative HPLC purification using a 10 minutes gradient from 42% to 55% MeOH in water as the eluent. Title compound (S)-5-((S)-5-(((2H-tetrazol-5-yl)methyl)carbamoyl)-3-cyclohexyl-2-oxoimidazolidin-1-yl)-4-((2S,3S)-2-(3-carboxypropanamido)-3-methylpentanamido)-5-oxopentanoic acid (C4) was obtained as an orange solid (10.5 mg, 28%). ¹H NMR (400 MHz, DMSO-d6) δ 0.70-0.83 (6H, m), 0.98-1.12 (2H, m), 1.20-1.45 (5H, m), 1.50-1.80 (6H, m), 1.95 (1H, m), 2.20-2.44 (5H, m), 3.22-3.43 (3H, m), 3.52-3.70 (2H, m), 4.16-4.20 (2H, m), 4.40-4.72 (2H, m), 5.40 (1H, m), 7.83 (1H, m), 8.10 (1H, m), 8.95 (1H, m), MS (LC/MS) m/z observed 635.93, expected 636.31 [M+H].

### Example C5

### (S)-5-((S)-5-(((2H-Tetrazol-5-yl)methyl)carbamoyl)-2-oxoimidazolidin-1-yl)-4-((2S,3S)-2-(3-carboxypropanamido)-3-methylpentanamido)-5-oxopentanoic acid

(S)-(−)-1-Z-2-Oxo-5-imidazolidinecarboxylic acid (2.0 g, 7.569 mmol, 1 eq.), DMAP (92.5 mg, 0.757 mmol, 0.1 eq.) and allyl alcohol (1.03 mL, 15.14 mmol, 2 eq.) were dissolved in CH₂Cl₂ (35 mL). The reaction was cooled to 0° C. and EDC (1.74 g, 9.083 mmol, 1.2 eq.) was added. The reaction was left at 0° C. for 1 h and stirred at RT for 16 hrs. The solvent was evaporated and the product was purified by normal phase column chromatography using 15% to 70% ethyl acetate in hexanes as the eluent to give (S)-5-allyl 1-benzyl 2-oxoimidazolidine-1,5-dicarboxylate as a white solid (1.70 g, 74%). ¹H NMR (400 MHz, CDCl₃) δ 3.42 (1H, dd, J=4, 7 Hz), 3.75 (1H, t, J=10 Hz), 4.53-4.63 (2H, m), 4.78 (1H, dd, J=4, 10 Hz), 5.20-5.33 (4H, m), 5.80 (1H, m), 6.10 (1H, s), 7.30-7.40 (5H, m), (LC/MS) m/z observed 304.85, expected 305.11 [M+H].

95% Dry NaH (147 mg, 6.145 mmol, 1.1 eq.) was carefully added to a solution of (S)-5-allyl 1-benzyl 2-oxoimidazolidine-1,5-dicarboxylate (1.7 g, 5.587 mmol, 1 eq.) in anhydrous THF (150 mL) at 0° C. under N₂. The reaction was left at 0° C. for 5 min and then allowed to warm to 10° C. and was stirred for an additional hour at 10° C. The reaction was added AcOH (5 mL) and the solvent was evaporated. The product was then purified by column chromatography using 15% to 65% ethyl acetate in hexanes as the eluent to give (S)-4-allyl 1-benzyl 2-oxoimidazolidine-1,4-dicarboxylate as a white solid (860.3 mg, 51%). ¹H NMR (400 MHz, CDCl₃) δ 4.06-4.17 (2H, m), 4.22 (1H, dd, J=5, 9 Hz), 4.67 (2H, m), 5.25-5.37 (4H, m), 5.83-5.93 (1H, m), 5.97 (1H, s), 7.32-7.44 (5H, m), (LC/MS) m/z observed 304.85, expected 305.11 [M+H].

A solution of (S)-4-allyl 1-benzyl 2-oxoimidazolidine-1,4-dicarboxylate (352 mg, 1.157 mmol, 1 eq.) in anhydrous THF (10 mL) was cooled to −50° C. under N₂. Potassium tert-butoxide (129.8 mg, 1.157 mmol, 1 eq.) was then added, followed by Boc-L-glutamic acid benzyl ester N-hydroxysuccinimide ester (581.6 mg, 1.157 mmol, 1 eq.) and the reaction mixture was slowly allowed to warm up to −10° C. and it was stirred at that temperature for 1 h. Analysis of the reaction mixture by TLC showed completion of the reaction. The reaction mixture was added AcOH (2 mL) and the solvent was evaporated. The residue was submitted to a reverse phase column chromatography using 10% to 80% methanol in water as the eluent to give (4S)-4-allyl 1-benzyl 3-((S)-5-(benzyloxy)-2-((tert-butoxycarbonyl)amino)-5-oxopentanoyl)-2-oxoimidazolidine-1,4-dicarboxylate as a colorless glass (448 mg, 62%). (LC/MS) m/z observed 645.91, expected 646.24 [M+Na] Compound was confirmed using LC/MS and moved to next step as it was.

To a solution of (4S)-4-allyl 1-benzyl 3-((S)-5-(benzyloxy)-2-((tert-butoxycarbonyl)amino)-5-oxopentanoyl)-2-oxoimidazolidine-1,4-dicarboxylate (448 mg, 0.718 mmol) in CH₂Cl₂ (25 mL) under N₂ was added Pd(PPh₃)₄ (166 mg, 0.144 mmol, 0.2 eq.) and morpholine (0.188 mL, 2.15 mmol, 3 eq.). The reaction was left at RT for 2 hrs and the solvent was evaporated. The residue was submitted to a reverse phase column chromatography but the product and triphenylphosphine co-eluted. The product was thus re-purified by normal phase column chromatography using 80% ethyl acetate in hexanes to elute triphenylphosphine oxide and 10% methanol in CH₂Cl₂ to elute (4S)-3-((S)-5-(benzyloxy)-2-((tert-butoxycarbonyl)amino)-5-oxopentanoyl)-1-((benzyloxy)carbonyl)-2-oxoimidazolidine-4-carboxylic acid that was obtained as a colorless glass (230 mg, 55%). MS (LC/MS) m/z observed 605.89, expected 606.21 [M+Na]. Compound was confirmed using LC/MS and moved to next step as it was.

(4S)-Benzyl 4-((2H-tetrazol-5-yl)methyl)carbamoyl)-3-((S)-5-(benzyloxy)-2-((tert-butoxycarbonyl)amino)-5-oxopentanoyl)-2-oxoimidazolidine-1-carboxylate was prepared from (4S)-3-((S)-5-(benzyloxy)-2-((tert-butoxycarbonyl)amino)-5-oxopentanoyl)-1-((benzyloxy)carbonyl)-2-oxoimidazolidine-4-carboxylic acid and (2H-tetrazol-5-yl)methyl-amine using method A in DMF but without HCl treatment. The reaction was stirred at RT for 16 hrs and then heated to 50° C. for 4 additional hours. MS (LC/MS) m/z observed 686.90, expected 687.25 [M+Na]. Compound was confirmed using LC/MS and moved to next step as it was.

(S)-Benzyl 4-(((2H-tetrazol-5-yl)methyl)carbamoyl)-3-((S)-5-(benzyloxy)-2-((2S,3S)-2-((tert-butoxycarbonyl)amino)-3-methylpentanamido)-5-oxopentanoyl)-2-oxoimidazolidine-1-carboxylate was prepared from (4S)-benzyl 4-(((2H-tetrazol-5-yl)methyl)carbamoyl)-3-((S)-5-(benzyloxy)-2-((tert-butoxycarbonyl)amino)-5-oxopentanoyl)-2-oxoimidazolidine-1-carboxylate and Boc-L-isoleucine using method A in DMF. MS (LC/MS) m/z observed 799.69, expected 800.33 [M+Na]. Compound was confirmed using LC/MS and moved to next step as it was.

(S)-Benzyl 4-(((2H-tetrazol-5-yl)methyl)carbamoyl)-3-((S)-5-(benzyloxy)-2-((2S,3S)-2-((tert-butoxycarbonyl)amino)-3-methylpentanamido)-5-oxopentanoyl)-2-oxoimidazolidine-1-carboxylate (33.4 mg, 0.0468 mmol) and succinic anhydride (7.0 mg, 0.07 mmol, 1.5 eq.) were suspended in CH₂Cl₂ (5 mL). NEt₃ (0.026 mL, 0.187 mmol, 4 eq.) was added and the reaction mixture was stirred at RT for 1 hour. It went to completion. The solvent was evaporated and the residue was dissolved in methanol (10 mL) containing AcOH (1 mL) and palladium on charcoal 10% by wt (10 mg) was added to the solution under N₂. The flask was then flushed with H₂ and H₂ was bubbled into the reaction mixture for 4 hrs. The flask was flushed with N₂ and the reaction mixture was filtered over celite. The solids were washed with methanol (3×15 mL) and the filtrate and washings were then concentrated to give a residue that was submitted to preparative HPLC purification using a 10 minutes gradient from 15% to 25% MeOH in water as the eluent. Title compound (S)-5-((S)-5-(((2H-tetrazol-5-yl)methyl)carbamoyl)-2-oxoimidazolidin-1-yl)-4-((2S,3S)-2-(3-carboxypropanamido)-3-methylpentanamido)-5-oxopentanoic acid (C5) was obtained as an orange solid (3.7 mg, 14%). ¹H NMR (400 MHz, DMSO-d6) δ 0.70-0.83 (6H, m), 1.08 (1H, m), 1.40 (1H, m), 1.70 (1H, m), 1.95 (1H, m), 2.20-2.55 (5H, m), 3.22-3.40 (3H, m), 3.65 (1H, m), 4.16-4.26 (2H, m), 4.50-4.72 (2H, m), 5.42 (1H, m), 7.78-7.92 (2H, m), 8.10 (1H, m), 8.85 (1H, m), MS (LC/MS) m/z observed 553.95, expected 554.23 [M+H].

### Example C6

### (S)—N-((2H-Tetrazol-5-yl)methyl)-1-cyclohexyl-3-(2-(2-cyclopentylacetamido)acetyl)-2-oxoimidazolidine-4-carboxamide

A solution of (S)-tert-butyl 1-cyclohexyl-2-oxoimidazolidine-4-carboxylate (235 mg, 0.876 mmol, 1 eq., from Example C4) in anhydrous THF (8 mL) was cooled to −50° C. under N₂. Potassium tert-butoxide (98.3 mg, 0.876 mmol, 1 eq.) was then added, followed by Boc-glycine N-hydroxysuccinimide ester (239.4 mg, 0.876 mmol, 1 eq.) and the reaction mixture was slowly allowed to warm up to −10° C. and it was stirred at that temperature for 1 h. The reaction mixture was added AcOH (1 mL) and the solvent was evaporated. The residue was submitted to a reverse phase column chromatography using 10% to 70% methanol in water as the eluent to give (S)-tert-butyl 3-(2-((tert-butoxycarbonyl)amino)acetyl)-1-cyclohexyl-2-oxoimidazolidine-4-carboxylate as a colorless glass (160.3 mg, 43%). (LC/MS) m/z observed 447.92, expected 448.24 [M+Na] Compound was confirmed using LC/MS and moved to next step as it was.

(S)-tert-Butyl 3-(2-((tert-butoxycarbonyl)amino)acetyl)-1-cyclohexyl-2-oxoimidazolidine-4-carboxylate (204.7 mg, 0.481 mmol) was treated with HCl (4N) in dioxane (15 ml) and water (5 mL) at RT for 4 hours. Both the Boc and tert-butyl groups were removed. The solvents were evaporated and the residue obtained was dissolved in dioxane (10 mL) and water (5 mL). Boc₂O (115.5 mg, 0.529 mmol, 1.1 eq.) and DIPEA were added (0.167 mL, 0.962 mmol, 2 eq.) and the reaction was left at RT for 10 minutes. The reaction mixture was then acidified to pH 4 with a citric acid (saturated solution) and the solvents were concentrated. The product was purified by reverse phase C18 chromatography using 10% to 50% methanol in water as the eluent to give (S)-3-(2-((tert-butoxycarbonyl)amino)acetyl)-1-cyclohexyl-2-oxoimidazolidine-4-carboxylic acid as a colorless glass (122.5 mg, 69%). (LC/MS) m/z observed 391.91, expected 392.18 [M+Na] Compound was confirmed using LC/MS and moved to next step as it was.

(S)-tert-Butyl (2-(5-(((2H-tetrazol-5-yl)methyl)carbamoyl)-3-cyclohexyl-2-oxoimidazolidin-1-yl)-2-oxoethyl)carbamate was prepared from (S)-3-(2-((tert-butoxycarbonyl)amino)acetyl)-1-cyclohexyl-2-oxoimidazolidine-4-carboxylic acid and (2H-tetrazol-5-yl)methyl-amine using method A in DMF but without HCl treatment. The reaction was stirred at RT for 16 hrs. MS (LC/MS) m/z observed 450.74, expected 451.24 [M+H]. Compound was confirmed using LC/MS and moved to next step as it was.

Title compound (S)—N-((2H-tetrazol-5-yl)methyl)-1-cyclohexyl-3-(2-(2-cyclopentylacetamido)acetyl)-2-oxoimidazolidine-4-carboxamide (C6) was prepared from (S)-tert-butyl (2-(5-(((2H-tetrazol-5-yl)methyl)carbamoyl)-3-cyclohexyl-2-oxoimidazolidin-1-yl)-2-oxoethyl)carbamate and cyclopentyl acetic acid using method A in 2:1 mixture DMF/CH₂Cl₂. ¹H NMR (400 MHz, DMSO-d6) δ 1.02-1.17 (3H, m), 1.22-1.41 (4H, m), 1.43-1.62 (6H, m), 1.63-1.79 (5H, m), 2.07-2.16 (3H, m), 3.27 (1H, dd, J=4, 10 Hz), 3.57 (1H, m), 3.68 (1H, t, J=10 Hz), 4.16 (1H, dd, J=5, 9 Hz), 4.47-4.56 (2H, m), 4.57-4.67 (2H, m), 7.98 (1H, t, J=6 Hz), 8.98 (1H, t, J=6 Hz), MS (LC/MS) m/z observed 460.95, expected 461.26 [M+H].

### Example D1

### General Kinetic Enzyme Assay Protocol

A specific 2× assay buffer was prepared for the enzyme to be tested (see Table 3 for final 1× assay buffer compositions). If the assay buffer included DTT, it was added immediately prior to running the assay. A 2× enzyme mix was prepared (see Table 4 for enzyme assay conditions) at 80 uL per well. Compounds were screened at one or two appropriate concentrations (to determine the percent inhibition at those concentrations) and/or a full dose response curve (typically 8 points, to identify the IC₅₀) in duplicate, triplicate, or higher replicates as needed. An appropriate control was also assessed in full dose response, in duplicate for each assay/plate. Background control wells consisted of 1× assay buffer, DMSO (5% v/v) and substrate. Positive control wells consisted of enzyme, DMSO (5% v/v) and substrate. Test compounds and control compounds were diluted in DMSO to 40× the final desired concentration. For example, a test compound may be tested in dose response, in serial, tripling dilution condition starting at 20 uM and ending at 9.1 nM (or any appropriate concentration range and dilution scheme). Control compounds were prepared similarly. Diluted compounds were prepared in a dilution plate and transferred to the reaction plate (96-well medium binding plate (Greiner Bio-One FLUOTRAC™)) to allow for the desired final concentrations when added to the enzyme with AB. After mixing, the reaction plate was placed on a shaker (at 300 RPM) for 5 min, followed by incubation (covered) on the bench, for 20 min. Plates were warmed to reaction temperature (see Table 3) for a total incubation time of 30 min. Plates so prepared were ready for addition of substrate and the subsequent reaction.

An appropriate substrate for each assay was prepared in advance at 2× the final desired concentration (see Table 3) in DMSO. The appropriate substrate mix was added to each appropriate well on the reaction plate, and the plate was read immediately in the TECAN plate reader (TECAN INFINITE® M1000 Pro), set to the correct wavelength as needed for each assay (see Table 4) using 25 cycles, kinetic interval of 1 min, number of reads per well of 20 with shaking set to 1 s, double orbital, 2 mm amplitude. For fluorescent assays the gain was set to optimal (50%).

TABLE 3
Assay Buffer Composition.

| Enzyme | Assay Buffer Composition |
|---|---|
| Caspase 1, 3, 4, 5, 7, 8*, 9 & 10/a | 50 mM HEPES pH 7.2 |
| (General caspase assay buffer) | 50 mM NaCl |
| | 0.1% (w/v) CHAPS |
| | 10 mM EDTA |
| | 5% (v/v) Glycerol |
| | 10 mM DTT |
| GzmB & Caspase 8 | 50 mM HEPES pH 7.5 |
| | 10% (w/v) sucrose |
| | 0.2% (w/v) CHAPS |
| | 5 mM DTT |

*Can also use GzmB assay buffer for the Caspase-8 assay; Assay buffer components were sourced as follows: HEPES, DTT, Glycerol and sucrose: Sigma-Aldrich, St. Louis, MO, USA, NaCl and EDTA: Fisher Scientific, Pittsburgh, PA, USA, CHAPS: Calbiochem, Billerica, MA, USA.

TABLE 4
Enzyme assay conditions.

| | | Substrate | | Ex/Em | Assay | |
|---|---|---|---|---|---|---|
| Enzyme | | | Conc. | λ* | Temp | Control |
| Name | Conc. | Name | (μM) | (nm) | (° C.) | Inhibitor |
| hGzmB | 10 nM | Ac-IEPD-AMC | 150 | 380/460 | 30 | Ac-IEPD-CHO |
| Caspase-1 | 6.25 mU/μl | YVAD-AFC | 25 | 400/505 | 37 | Z-VAD-FMK |
| Caspase-3 | 6.25 mU/μl | Ac-DEVD-AMC | 20 | 380/460 | 37 | Z-VAD-FMK |
| and | | | | | | |
| Caspase 7 | | | | | | |
| Caspase-4 | 3.125 mU/ul | Ac-WEHD-AFC | 100 | 400/505 | 37 | Z-WEHD-FMK |
| and | | | | | | |
| Caspase-5 | | | | | | |
| Caspase-8 | 3.125 mU/ul | Ac-IEPD-AMC | 75 | 380/460 | 30 | Ac-IEPD-CHO |
| Caspase-9 | 3.125 mU/ul | LEHD-AFC | 50 | 400/505 | 37 | Q-LEHD-Oph |
| Caspase-10/a | 6.25 mU/μl | Ac-IETD-AMC | 100 | 400/505 | 30 | Ac-AEVD-CHO |

*Ex/Em λ is the excitation and emission wavelengths at which to measure fluorescence. Enzyme and substrate concentrations are the final concentrations in the well. Note that most protocols require preparing 2X enzyme and substrate mixes, as they are diluted 2-fold in the well.

Enzymes were sourced as follows: hGzmB, Froelich Lab, Northshore University Health Systems Research Institute, Evanston, Ill., USA; Caspases, Biovision Inc., Milpitas, Calif., USA. Substrates were sourced as follows: Ac-IEPD-AMC, California Peptide Research Inc., Napa, Calif., USA; YVAD-AFC, Biovision Inc., Milpitas, Calif., USA; Ac-DEVD-AMC, LEHD-AFC, AC-WEHD-AFC and Ac-IETD-AMC, Enzo Life Sciences Inc, Farmingdale, N.Y., USA. Control inhibitors were sourced as follows: Ac-IEPD-CHO, Ac-WEHD-FMK and Q-LEHD-Oph, Biovision Inc., Milpitas, Calif., USA; Z-VAD-FMK, R&D Systems, Minneapolis, Minn., USA; and Ac-AEVD-CHO, Enzo Life Sciences Inc, Farmingdale, N.Y., USA.

### Example D2

### Human Granzyme B Enzymatic Inhibition Assay

An in vitro fluorogenic detection assay for assessing the IC₅₀ and/or percent inhibition at a given concentration of inhibitors against human Granzyme B (hGzmB) enzyme was performed as described in Example D1. When appropriate, percent inhibition data was collected and fitted to generate IC₅₀ data using GraphPad Prism 5 (GraphPad Software, La Jolla Calif. USA, www.graphpad.com) and its non-linear regression analysis tools or other equivalent tools.

Select compounds of Examples C1-C6 exhibited inhibitory activity against hGzmB. Each of the compounds identified in Table 1 exhibited Granzyme B inhibitory activity.

In certain embodiments, select compounds exhibited IC₅₀<50,000 nM. In other embodiments, select compounds exhibited IC₅₀<10,000 nM. In further embodiments, select compounds exhibited IC₅₀<1,000 nM. In still further embodiments, select compounds exhibited IC₅₀<100 nM. In certain embodiments, select compounds exhibited IC₅₀ from 10 nM to 100 nM, preferably from 1 nM to 10 nM, more preferably from 0.1 nM to 1 nM, and even more preferably from 0.01 nM to 0.1 nM.

### Example D3

### Human Caspase Enzymatic Inhibition Assay

In vitro fluorogenic detection assays for assessing the IC₅₀ and/or percent inhibition at a given concentration of inhibitors, against a set of human Caspase enzymes, was performed as described in Example D1. Representative compounds do not significantly inhibit any caspase enzyme tested at a concentration of 50 μM.

In certain embodiments, the compounds exhibited less than 50% inhibition at 50 μM. In other embodiments, the compounds exhibited greater than 50% inhibition at 50 μM, but less than 10% inhibition at 25 μM.

### Example D4

### General Kinetic Enzyme Assay Protocol (384 Well)

A specific 2× assay buffer was prepared for the enzyme to be tested (see Table 4 for final 1× assay buffer compositions). If the assay buffer included DTT, it was added immediately prior to running the assay. A 2× enzyme mix was prepared (see Table 3 for enzyme assay conditions) at 26 uL per well. Compounds were screened at one or two appropriate concentrations (to determine the percent inhibition at those concentrations) and/or a full dose response curve (typically 12 points, to identify the IC₅₀) in duplicate, triplicate, or higher replicates as needed. An appropriate control was also assessed in full dose response, in duplicate for each assay/plate. Background control wells consisted of 1× assay buffer and substrate. Positive control wells consisted of enzyme (no DMSO) and substrate. Test compounds and control compounds were diluted in 1× Assay Buffer to 15× the final desired concentration. For example, a test compound may be tested in dose response, in serial, tripling dilution condition starting at 20 uM and ending at 0.1 nM (or any appropriate concentration range and dilution scheme). Control compounds were prepared similarly. Diluted compounds were prepared in a dilution plate and transferred to the reaction plate (384-well medium binding plate (Greiner Bio-One FLUOTRAC™)) to allow for the desired final concentrations when added to the enzyme with AB. After mixing, the reaction plate was placed on a shaker (at 300 RPM) for 5 min, followed by incubation (covered) on the bench, for 20 min. Plates were warmed to reaction temperature (see Table 5) for 5 mins for a total incubation time of 30 min. Plates so prepared were ready for addition of substrate and the subsequent reaction.

An appropriate substrate for each assay was prepared in advance at 2× the final desired concentration (see Table 5) in assay buffer. 30 uL of the appropriate substrate mix was added to each appropriate well on the reaction plate, and the plate was read immediately in the TECAN plate reader (TECAN INFINITE® M1000 Pro), set to the correct wavelength as needed for each assay (see Table 6) using 15 cycles, kinetic interval of 1 min, number of reads per well of 20 with shaking set to 1 s, double orbital, 2 mm amplitude. For fluorescent assays the gain was set to optimal (100% with gain regulation) for all assays except human GzmB which was set to 85 (with the z set at 23000 um).

TABLE 5
Assay Buffer Composition.

| Enzyme | Assay Buffer Composition |
|---|---|
| Caspase 1, 3, 4, 5, 7, 8*, 9 & 10/a | 50 mM HEPES pH 7.2 |
| (General caspase assay buffer) | 50 mM NaCl |
| | 0.1% (w/v) CHAPS |
| | 10 mM EDTA |
| | 5% (v/v) Glycerol |
| | 10 mM DTT |
| GzmB & Caspase 8 | 50 mM HEPES pH 7.5 |
| | 0.2% (w/v) CHAPS |
| | 5 mM DTT |
| Cathepsin G | 320 mM Tris-HCL pH 7.4 |
| | 3.2M NaCl |

*Can also use GzmB assay buffer for the Caspase-8 assay; Assay buffer components were sourced as follows: HEPES, DTT, Glycerol and sucrose: Sigma-Aldrich, St. Louis, MO, USA, NaCl and EDTA: Fisher Scientific, Pittsburgh, PA, USA, CHAPS: Calbiochem, Billerica, MA, USA.

TABLE 6
Enzyme assay conditions.

| | | Substrate | | Ex/Em | Assay | |
|---|---|---|---|---|---|---|
| Enzyme | | | Conc. | λ* | Temp | Control |
| Name | Conc. | Name | (μM) | (nm) | (° C.) | Inhibitor |
| hGzmB | 10 nM | Ac-IEPD-AMC | 50 | 380/460 | 30 | V2248 |
| Caspase-1 | 12.5 mU/μL | YVAD-AFC | 5 | 400/505 | 37 | Z-VAD-FMK |
| Caspase-3 | 0.8 mU/μL & | Ac-DEVD-AMC | 40 & 5 | 380/460 | 37 | Z-VAD-FMK |
| and | 1.5 mU/μL | | | | | |
| Caspase 7 | | | | | | |
| Caspase-4 | 3.125 mU/uL & | Ac-WEHD-AFC | 40 & 100 | 400/505 | 37 | Z-WEHD-FMK |
| and | 1.5 mU/uL | | | | | |
| Caspase-5 | | | | | | |
| Caspase-8 | 4 mU/uL | Ac-IEPD-AMC | 80 | 380/460 | 37 | Ac-IEPD-CHO |
| Caspase-9 | 2 mU/uL | LEHD-AFC | 50 | 400/505 | 37 | Q-LEHD-Oph |
| Caspase-10/a | 3 mU/μL | Ac-IETD-AMC | 10 | 400/505 | 37 | Ac-AEVD-CHO |
| Cathepsin G | 200 nM | Suc-AAPF-pNA | 200 uM | 410 | 25 | Cat G |
| | | | | absorbance | | inhibitor |
| Human | 0.125 ug/mL | MeOSuc- | 50 | 384/500 | 37 | Sivelestat |
| Neutrophil | | AAPF-AFC | | | | |
| Elastase | | | | | | |

*Ex/Em λ is the excitation and emission wavelengths at which to measure fluorescence. Enzyme and substrate concentrations are the final concentrations in the well. Note that most protocols require preparing 2X enzyme and substrate mixes, as they are diluted 2-fold in the well.

Enzymes were sourced as follows: hGzmB, Froelich Lab, Northshore University Health Systems Research Institute, Evanston, Ill., USA; Caspases and Elastase, Biovision Inc., Milpitas, Calif., USA; Cathepsin G, Athens Research and Technologies, Athens, Ga., USA. Substrates were sourced as follows: Ac-IEPD-AMC, California Peptide Research Inc., Napa, Calif., USA; YVAD-AFC and MeOSuc-AAPF-AFC Biovision Inc., Milpitas, Calif., USA; LEHD-AFC and Suc-AAPF-pNA Millipore, Billerica Mass., USA. Ac-DEVD-AMC, AC-WEHD-AFC and Ac-IETD-AMC, Enzo Life Sciences Inc, Farmingdale, N.Y., USA. Control inhibitors were sourced as follows: Ac-IEPD-CHO, Ac-WEHD-FMK, Q-LEHD-Oph and CatG inhibitor, Biovision Inc., Milpitas, Calif., USA; Z-VAD-FMK, R&D Systems, Minneapolis, Minn., USA; and Ac-AEVD-CHO, Enzo Life Sciences Inc, Farmingdale, N.Y., USA. Sivelestat, Tocris Bioscience, Bristol, UK.

### Example D5

### Human Granzyme B Enzymatic Inhibition Assay

An in vitro fluorogenic detection assay for assessing the IC₅₀ and/or percent inhibition at a given concentration of inhibitors against human Granzyme B (hGzmB) enzyme was performed as described in Example D4. When appropriate, percent inhibition data was collected and fitted to generate IC₅₀ data using GraphPad Prism 5 (GraphPad Software, La Jolla Calif. USA, www.graphpad.com) and its non-linear regression analysis tools or other equivalent tools.

Select compounds of Examples C1-C6 exhibited inhibitory activity against hGzmB. Each of the compounds of the invention identified in Table 1 exhibited Granzyme B inhibitory activity.

In certain embodiments, select compounds exhibited IC₅₀<50,000 nM. In other embodiments, select compounds exhibited IC₅₀<10,000 nM. In further embodiments, select compounds exhibited IC₅₀<1,000 nM. In still further embodiments, select compounds exhibited IC₅₀<100 nM. In certain embodiments, select compounds exhibited IC₅₀ from 10 nM to 100 nM, preferably from 1 nM to 10 nM, more preferably from 0.1 nM to 1 nM, and even more preferably from 0.01 nM to 0.1 nM.

### Example D6

### Human Caspase Enzymatic Inhibition Assay

In vitro fluorogenic detection assays for assessing the IC₅₀ and/or percent inhibition at a given concentration of inhibitors, against a set of human Caspase enzymes, was performed as described in Example D4. Representative compounds do not significantly inhibit any caspase enzyme tested at a concentration of 50 μM.

In certain embodiments, the compounds exhibited less than 50% inhibition at 50 μM. In other embodiments, the compounds exhibited greater than 50% inhibition at 50 μM, but less than 10% inhibition at 25 μM.

### Example D7

### Inhibition of Fibronectin Cleavage by GzmB

Black, 96 well high-binding assay plates (Griener Bio-one) were treated overnight at 4° C. with 40 uL of 8 ug/mL Hilyte Fluor 488 labeled Fibronectin (Cytoskeleton, Inc). After fibronectin coating, plates were washed 3 times in buffer (20 mM Tris-HCl, pH 7.4, 20 mM NaCl) then once with granzyme B assay buffer (50 mM HEPES, pH 7.5, 0.1% CHAPS). After washing, 50 uL of granzyme B assay buffer was added to each fibronectin-coated well. In a separate non-binding 96 well assay plate 5 uL of 20× inhibitor serial dilution stocks were added to 45 uL of 2.22×GzmB mix to establish inhibition (enzyme/inhibitor mixes were all prepared in granzyme B assay buffer and were incubated first at room temperature for 20 minutes, then at 30° C. for another 10 minutes). After incubation, 50 uL of this 2× enzyme/inhibitor mix was added to the corresponding coated well to initiate fibronectin cleavage (20 nM final granzyme B concentration, 8-point inhibitor dilution series starting at 50 uM). The assay was conducted at 30° C. in the TECAN plate reader (TECAN INFINITE® M1000 Pro), which was programmed to monitor the kinetic fluorescence polarization signal (filter set Ex/Em 470 nm/527 nm) with readings taken every minute, for 1 hour. Proteolytic activity was evaluated as the rate of fluorescence enhancement in the parallel emission over the linear range of the reaction. % Inhibition values were calculated from assay controls and the resulting data is shown in Table 7.

TABLE 7
Inhibition of Fibronectin Cleavage by GzmB Results.

| | Percent Inhibition at Inhibitor Concentration | | |
|---|---|---|---|
| Compound | 50 uM | 5. 56 uM | 0.62 uM |
| C1 | 86% | 69% | 39% |
| C2 | 96% | 91% | 75% |

### Example 1

### Method for Evaluating Granzyme B Inhibitor Permeation

Compound A permeation through skin was assessed ex vivo by using pig ear skin and a Franz Diffusion Cell System (PermeGear®).

Fresh pig ears were obtained from female Yorkshire/Landrace crosses, between 3.5-5 months old, at Jack Bell Research Centre, Vancouver. Immediately after surgery, pig ears were removed and placed in a double Ziploc® bag and stored at −20° C. freezer until shipment to laboratory site on ice via courier the next day. Upon arrival at the site, pig ears were stored in a segregated container, labeled with harvest and shipment date, in −20° C. freezer and were used prior to their expiry date of 5 weeks. Pig skin was prepared according to “Preparation of Pig Skin Samples for ex vivo Permeation Assays.” Briefly, one day prior to the study, frozen ears were thawed washed with deionized water and blotted dry. After trimming off hair, surface skin was separated from cartilage and fat, and hole-punched into coin shape samples to fit dimensions of Franz cells. Pig ear samples were stored in a petri dish, sealed with parafilm tape, in double Ziploc® bags at −20° C. overnight prior to the study on the following day.

Set up of the diffusion cell system was performed according to “Setup, Operation and Cleaning Procedure for Franz Diffusion Cell System.” Prior to application of formulations, skin integrity of each skin sample was determined by measuring the electrical resistance. The electrical resistance was measured with a Model 878B Dual Display LCR Meter (BK Precision®) connected to two stainless steel electrodes, using a setting of 1 KHz. Measurements were taken at least 30 minutes after mounting skin samples to ensure temperature and humidity equilibration. The donor and receptor chamber were filled with 300 μL and 5 mL of pH 7.4 phosphate buffered saline (PBS), respectively, and both electrodes were immersed in solution without touching the skin membrane. Any skin membrane giving an electrical resistance below 4.0 kΩ was discarded and replaced.

Throughout the permeation assay, all cells were maintained at 32° C. and sink condition was maintained in the receptor fluid. Receptor chambers were filled with 4.75 mL of receptor fluid (pH 7.4 PBS) and were kept stirring for the entire duration of the experiment. Skin samples were mounted above the receptor chamber, in direct contact with the receptor fluid, and then donor chambers were mounted above the skin. Any air bubbles between the skin tissue and the receptor fluid were carefully removed prior to application of formulations. The appropriate formulation (300 μL) was added to the donor chamber and the opening of the cell was covered with Parafilm® to prevent evaporation of the test article. Three skin samples per formulation (N) were tested. Three components (receptor fluid, applied formulation and skin samples) were sampled for subsequent analysis. Receptor fluid was sampled at 3 and 6 hours (without volume replacement) and stored at −20° C. until analysis. At the end of the experiment, the applied formulation was recovered from the donor chamber and recorded as the unabsorbed dose. The skin was then blotted dry with a Kimwipe® and tape-stripped skin (12 tape strips) was obtained. The first tape strip (3M scotch tape) was used to remove excess of formulation remaining on the skin and was discarded. Then a total of 11 tape strips were used to partially remove the stratum corneum. These tape strips were kept in labeled glass vial. Then using a hole-puncher the section of skin exposed to the formulation was removed and the corresponding weight of the skin sample was recorded. All samples were stored at −20° C. until analysis.

Analytical Methods.

UPLC-MS/MS technology was used to separate and quantify Compound A from skin tissue extract and from receptor chamber fluid (PBS, pH 7.4). A Waters Acquity UPLC-TQD system was used for this purpose. Separation was performed using gradient elution on a 2.1×50 mm C18 column, with 1.7 μm particle size. Positive electrospray ionization was applied to the mass spectrometer source and the analyzer was operated in multiple reaction monitoring mode (MRM).

Compound A was extracted from skin tissue by homogenization in water and in acetonitrile, followed by high frequency centrifugation. The supernatant was then highly diluted in mobile phase (dilution factor 50 to 200) for analysis against neat standards prepared in mobile phase. The mobile phase sample diluent was a mixture of 75% of 10 mM ammonium acetate adjusted with ammonium hydroxide buffer to pH 8.8 and 25% acetonitrile.

For receptor fluid, the buffer was diluted by a factor of two using acetonitrile, vortexed, and analyzed directly. Standards and quality control samples (QC's) were prepared in the same matrix as the test samples (PBS/acetonitrile). Table 8 provides a summary of the analytical method parameters.

TABLE 8
Summary of Method Evaluation Results.

| | Mobile Phase Diluent | |
|---|---|---|
| | (75% Ammonium | |
| | Acetate/Ammonium | |
| | Hydroxide pH 8.7, | |
| Parameter | 25% ACN) | pH 7.4 PBS |
| Selectivity | No interference | No interference |
| | observed | observed |
| Linear Range (1/x weighting) | 2.5-100 ng/mL | 1-200 ng/mL |
| Limit of Quantitation (LOQ) | <2.5 ng/mL | 1 ng/mL |
| Average Accuracy (% | Not calculated | 6.5% |
| deviation) at LOQ | | |
| Average Accuracy | 4.2% | 0.9% |
| (% deviation) mid-calibration | | |
| range | | |
| Precision (% RSD) at LOQ | Not calculated | 7.2% |
| Avg. Extraction Recovery (%) | N/A | N/A |

Results.

Samples for determination of Compound A concentration in the skin were collected after 6 hours of exposure to formulations. Concentration measurements of Compound A in reservoir fluid were performed on samples collected after 3 and 6 hours of exposure. The values for individual samples are provided in Table 9.

Compound A concentrations measured in tissue and reservoir fluid for each sample.

TABLE 9
Amount of Compound A in tape-stripped skin normalized to skin mass
after 6 hours exposure to formulations. Amount of Compound A in
reservoir fluid after 3 and 6 hours of exposure to formulations.

| | Time | | Skin | Reservoir fluid |
|---|---|---|---|---|
| | point | | concentration | concentration |
| Formulations | (h) | Replicate | (ng/g) | (ng/mL) |
| pH5 acetate | 3 | 1 | — | ND |
| buffer + 20% PG | | 2 | — | ND |
| | | 3 | — | ND |
| | 6 | 1 | 4785 | ND |
| | | 2 | 10012 | ND |
| | | 3 | 6515 | 1.1 |
| pH5 acetate | 3 | 1 | — | ND |
| buffer + 10% Urea | | 2 | — | ND |
| | | 3 | — | BLOQ |
| | 6 | 1 | 5588 | ND |
| | | 2 | 19422 | ND |
| | | 3 | 2943 | 2.6 |
| pH5 acetate | 3 | 1 | — | ND |
| buffer + 15% Tw80 | | 2 | — | 1.8 |
| | | 3 | — | ND |
| | 6 | 1 | 4937 | BLOQ |
| | | 2 | 1968 | BLOQ |
| | | 3 | 4442 | ND |
| pH5 acetate | 3 | 1 | — | 43.3 |
| buffer + 10% DMI | | 2 | — | ND |
| | | 3 | — | ND |
| | 6 | 1 | 13975 | 68.5 |
| | | 2 | 6214 | BLOQ |
| | | 3 | 6311 | BLOQ |
| pH5 acetate | 3 | 1 | — | ND |
| buffer + 10% TC | | 2 | — | ND |
| | | 3 | — | ND |
| | 6 | 1 | 5869 | ND |
| | | 2 | 8886 | BLOQ |
| | | 3 | 1629 | ND |
| Water + 20% NMP | 3 | 1 | — | BLOQ |
| | | 2 | — | ND |
| | | 3 | — | ND |
| | 6 | 1 | 4313 | 1.1 |
| | | 2 | 4985 | ND |
| | | 3 | 5241 | BLOQ |

Skin Concentrations.

The concentration of Compound A in tape-stripped skin after 6 hours of exposure to formulations is summarized in Table 10. The number of skin samples used per formulation was 3 for each group. Average skin concentrations were 3700-9300 ng/g.

TABLE 10
Amount of Compound A in tape-stripped skin normalized to skin mass
after 6 hour exposure to formulation.

| | Compound A in | | | | |
|---|---|---|---|---|---|
| | tape-stripped | | | | |
| | skin samples (ng/g) | | | | St. |
| Formulation ID | 1 | 2 | 3 | Average | Dev. |
| pH5 acetate buffer + 20% | 4784 | 10012 | 6515 | 7104 | 2663 |
| Propylene Glycol (PG) | | | | | |
| pH5 acetate buffer + 10% | 5588 | 19422 | 2943 | 9317 | 8849 |
| Urea | | | | | |
| pH5 acetate buffer + 15% | 4937 | 1968 | 4442 | 3782 | 1590 |
| Tween 80 | | | | | |
| pH5 acetate buffer + 10% | 13975 | 6214 | 6311 | 8833 | 4453 |
| Dimethyl Isosorbide | | | | | |
| (DMI) | | | | | |
| pH5 acetate buffer + 10% | 5869 | 8886 | 1629 | 5461 | 3645 |
| Transcutol ® (TC) | | | | | |
| Water + 20% N-methy1- | 4313 | 4985 | 5241 | 4846 | 479 |
| 2-pyrollidone (NMP) | | | | | |

Receptor Fluid Concentrations.

Formulations tested ex vivo contained different amount of Compound A according to the solubility of the compound in the corresponding vehicle. To better analyze the effect of PE on the drug permeation, values found in skin were not only normalized to skin mass but also to the corresponding drug concentration in formulation.

Table 11 summarizes the concentrations of Compound A detected in the receptor fluid after 3 and 6 hours of exposure to formulations. The number of receptor fluid samples analyzed per formulation was 3 for each time point. In general, drug concentration in receptor fluids was below limit of quantitation (BLOQ) or non-detected (ND) at both times of exposure time. Only one out of three samples containing 10% DMI showed considerable amount of drug in receptor fluid.

TABLE 11
Concentration of Compound A in receptor fluid (ng/mL) after 3 and
6 hours of exposure to formulations.

| | Exposure | Compound A in receptor fluid (ng/mL) | | | | | | | | |
|---|---|---|---|---|---|---|---|---|---|---|
| Formulation | Time (h) | 1 | 2 | 3 | 4 | 5 | 6 | 7 | 8 | 9 |
| pH5 acetate | 3 | ND | ND | ND | | | | | | |
| buffer + 20% PG | 6 | ND | ND | 1.1 | | | | | | |
| pH5 acetate | 3 | | | | ND | ND | BLOQ | | | |
| buffer + 10% Urea | 6 | | | | ND | ND | 2.6 | | | |
| pH5 acetate | 3 | | | | | | | ND | 1.8 | ND |
| buffer + 15% Tw80 | 6 | | | | | | | BLOQ | BLOQ | ND |
| pH5 acetate | 3 | 43.3 | ND | ND | | | | | | |
| buffer + 10% DMI | 6 | 68.5 | BLOQ | BLOQ | | | | | | |
| pH5 acetate | 3 | | | | ND | ND | ND | | | |
| buffer + 10% TC | 6 | | | | ND | BLOQ | ND | | | |
| Water + 20% NMP | 3 | | | | | | | BLOQ | ND | ND |
| | 6 | | | | | | | 1.1 | ND | BLOQ |

^{†}BLOQ: Compound A was detected in the sample below the assays limit of quantitation, 1 ng/mL in PBS.
ND: Not detected.

### Example 2

### Representative Granzyme B Inhibitor Formulation and Performance Properties

In this example, the preparation and performance properties of a representative Granzyme B inhibitor formulation is described. The representative Granzyme B inhibitor formulation includes Compound 1A and was formulated as a gel at either 3.6 or 10.0 mg/mL Compound 1A based on the volume of the gel.

Procedure for Making the Base Vehicle

The base vehicle included 20% PG, 0.2% methyl paraben, 0.02% propyl paraben, in acetate buffer (10 mM, pH 5). The base vehicle was prepared by mixing 20% of PG with acetate buffer (10 mM, pH 5). An excess amount of methyl paraben and propyl paraben (0.2% methyl paraben/0.02% propyl paraben) was added to the solution, stirred overnight (>8 hours) at room temperature. The pH of the solution was adjusted to pH 5 with 1M HCl. The final mixture was filter via 0.45 um filter.

Procedure for Making the Gel Formulations:

For the pre-clinical lab scale (non-sterile), a 13 mg/mL formulation was prepared by adding Compound 1A into the vehicle (20% PG, 0.2% methyl paraben, 0.02% propyl paraben, acetate buffer (10 mM, pH 5), prepared as described above) and sonicated for 1 hour. 1% Carbopol 940 NF was added to the formulation and the mixture was stirred for 24 hours at room temperature in order to fully hydrate the Carbopol. After 24 hours, the pH was adjusted to pH 6.0±0.2 with triethanolamine. The final formulation is a colorless transparent gel. The formulation is physically and chemically stable with over 90% Compound 1A recovery by UPLC-UV up to 1 month at refrigeration storage conditions (2-8° C.).

Additional representative gels were prepared as described above with Compound 1A up to and including 20 mg/mL. In certain embodiments, Compound 1A and Carbopol were separately prepared, each titrated to pH 6 with triethanolamine, and then combined.

The representative formulations can be sterilized. For example, the hydrated Carbopol mixture can be sterilized via autoclave process and the Compound 1A solution can be filtered via 0.22 um filtration, the combination process can be performed in a sterilized environment.

Performance In Vivo

The in vivo performance for a representative gel prepared as described above comprising Compound 1A (3.6 mg/mL) dissolved in a vehicle comprising PG (20% w/w), Carbopol 940 (0.5% w/v), 0.2% w/w methyl paraben, 0.02% w/w propyl paraben, acetate buffer (10 mM, pH 5) adjusted to pH 6.0 with triethanolamine had a performance comparable to that of another representative formulation (Compound 1 (3.6 mg/mL) dissolved in a vehicle comprising PG (20% w/w), Carbopol 940 (0.5% w/v), 0.2% w/w methyl paraben, 0.02% w/w propyl paraben, acetate buffer (10 mM, pH 5) adjusted to pH 6.0 with triethylamine), which data is shown in FIGS. 5A-6C.

Franz Cell Ex Vivo Skin Permeation

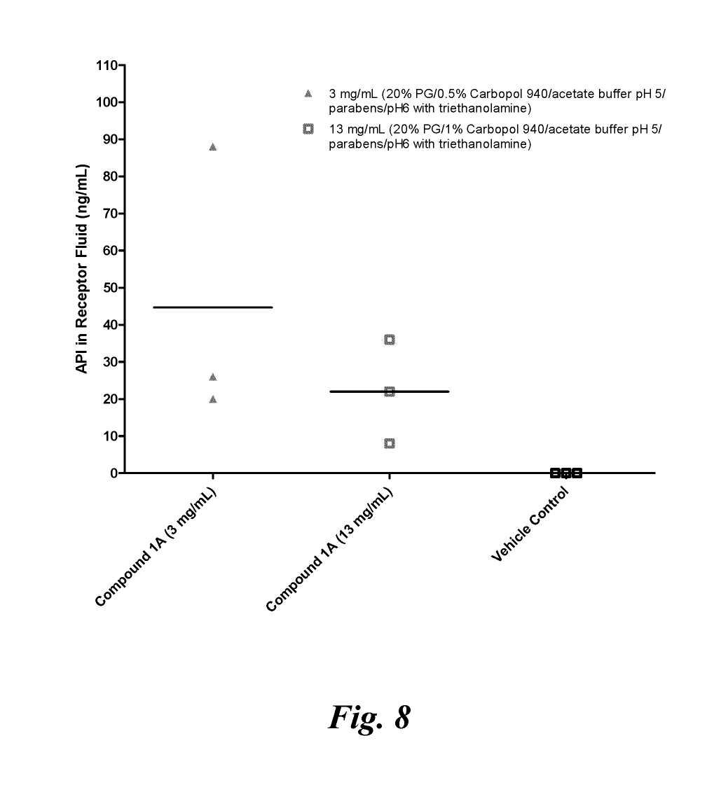

Franz cell ex vivo pig skin permeation data was generated using a 13 mg/mL Compound 1A formulation prepared as described above. The skin permeation was determined as described above in Example 1. The skin permeation data are shown in FIGS. 7 and 8. FIG. 7 shows the amount of Compound 1A (formulated at 3 and 13 mg/mL) in the tape-stripped skin sample compared to vehicle control (no active). FIG. 8 shows the amount of Compound 1A (formulated at 3 and 13 mg/mL) in receptor fluid compared to vehicle control.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A formulation for burn wound healing, comprising a compound having Formula (I): 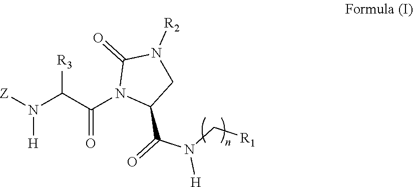
stereoisomers, tautomers, or pharmaceutically acceptable salts thereof, wherein:
R₁ is a heteroaryl group selected from
(a) 1,2,3-triazolyl, and
(b) 1,2,3,4-tetrazolyl;
n is 1 or 2;
R₂ is selected from hydrogen, C1-C6 alkyl, and C3-C6 cycloalkyl;
R₃ is selected from
(a) hydrogen,
(b) C₁-C₄ alkyl optionally substituted with a carboxylic acid, carboxylate, or carboxylate C₁-C₈ ester group (—CO₂H, —CO₂⁻, —C(═O)OC₁-C₈), an amide optionally substituted with an alkylheteroaryl group, or a heteroaryl group;
Z is an acyl group selected from the group
(a) 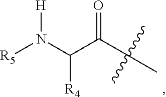
and
(b) 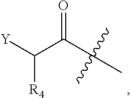
wherein
Y is hydrogen, heterocycle, —NH₂, or C₁-C₄ alkyl;
R₄ is selected from
(i) C₁-C₁₂ alkyl,
(ii) C₁-C₆ heteroalkyl optionally substituted with C₁-C₆ alkyl,
(iii) C₃-C₆ cycloalkyl,
(iv) C₆-C₁₀ aryl,
(v) heterocyclyl,
(vi) C₃-C₁₀ heteroaryl,
(vii) aralkyl, and
(viii) heteroalkylaryl;
R₅ is heteroaryl or —C(═O)—R₁₀, wherein R₁₀ is selected from
(i) C₁-C₁₂ alkyl optionally substituted with C₆-C₁₀ aryl, C₁-C₁₀ heteroaryl, amino, or carboxylic acid,
(ii) heteroalkyl optionally substituted with C₁-C₆ alkyl or carboxylic acid,
(iii) C₃-C₆ cycloalkyl optionally substituted with C₁-C₆ alkyl, optionally substituted C₆-C₁₀ aryl, optionally substituted C₃-C₁₀ heteroaryl, amino, or carboxylic acid,
(iv) C₆-C₁₀ aryl optionally substituted with C₁-C₆ alkyl, optionally substituted C₆-C₁₀ aryl, optionally substituted C₃-C₁₀ heteroaryl, amino, or carboxylic acid,
(v) heterocyclyl,
(vi) C₃-C₁₀ heteroaryl,
(vii) aralkyl, and
(viii) heteroalkylaryl, and
a pharmaceutically acceptable carrier.

2. The formulation of claim 1, wherein the compound is selected from the group consisting of C1, C2, C3, C4, C5, C6, and stereoisomers, tautomers, or pharmaceutically acceptable salts thereof.

3. The formulation of claim 1, wherein the compound is 4-(((2S,3S)-1-((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)amino)-4-oxobutanoic acid or a pharmaceutically acceptable salt thereof.

4. The formulation of any one of claims 1-3 further comprising a skin penetration enhancer.

5. The formulation of claim 4, wherein the skin penetration enhancer is propylene glycol.

6. The formulation of claim 4 further comprising a viscosity enhancer.

7. The formulation of claim 6, wherein the viscosity enhancer is a crosslinked polyacrylate polymer.

8. The formulation of any one of claims 1-7 having a pH of from about 4 to about 7.4.

9. The formulation of any one of claims 1-7 having a pH of about 6.0.

10. The formulation of any one of claims 1-9 in the form of a gel comprising from about 0.5 to about 20 mg/mL of a compound of formula (I).

11. The formulation of any one of claims 1-9 in the form of a gel comprising about 10 mg/mL of a compound of formula (I).

12. A method of treating a burn in a subject, comprising administering a therapeutically effective amount of a formulation of any one of claims 1-11 to a subject in need thereof.

13. The method of claim 12, wherein the formulation is topically administered.

14. The method of claim 12, wherein the formulation is administered by injection.

15. A method of healing a burn wound in a subject, comprising administering therapeutically effective amount of a formulation of any one of claims 1-11 to a subject in need thereof.

16. The method of claim 15, wherein the formulation is topically administered.

17. The method of claim 15, wherein the formulation is administered by injection.

18. A method for reducing or preventing the expansion of the zone of stasis of a burn wound in a subject, comprising administering a therapeutically effective amount of formulation of any one of claims 1-11 to a subject in need thereof.

19. The method of claim 18, wherein the formulation is topically administered.

20. The method of claim 18, wherein the formulation is administered by injection.

21. A method for intradermal delivery of a Granzyme B inhibitor to a subject, comprising administering a formulation of any one of claims 1-11 to a subject in need thereof.

22. The method of claim 21, wherein the formulation is topically administered.

23. The method of claim 21, wherein the formulation is administered by injection.
